SEQUENCE LISTING (34 sequences)

SEQ ID NO: 1 - protein, 9 aa, synthetic construct
PLAPDKTHT

SEQ ID NO: 2 - protein, 4 aa, synthetic construct
PLAP

SEQ ID NO: 3 - protein, 20 aa, synthetic construct
GGGGSGGGGSGGGGSGGGGS

SEQ ID NO: 4 - protein, 7 aa, synthetic construct
EKSYGPP

SEQ ID NO: 5 - protein, 5 aa, synthetic construct
DKTHT

SEQ ID NO: 6 - protein, 11 aa, synthetic construct
CPPCPAPELLG

SEQ ID NO: 7 - protein, 15 aa, synthetic construct
PLAPCPPCPAPELLG

SEQ ID NO: 8 - protein, 16 aa, synthetic construct
DKTHTCPPCPAPELLG

SEQ ID NO: 9 - protein, 18 aa, synthetic construct
EKSYGPPCPPCPAPELLG

SEQ ID NO: 10 - protein, 5 aa, synthetic construct
KHAMG

SEQ ID NO: 11 - protein, 17 aa, synthetic construct
AIDWSGGSTYYADSVKG

SEQ ID NO: 12 - protein, 20 aa, synthetic construct
DSYTDYAQLWLPELESEYDY

SEQ ID NO: 13 - protein, 5 aa, synthetic construct
SYTMG

SEQ ID NO: 14 - protein, 17 aa, synthetic construct
AISWSAGRTYYADSVKG

SEQ ID NO: 15 - protein, 22 aa, synthetic construct
EEAPDWAPIDCSGYGCLSLYDY

SEQ ID NO: 16 - protein, 129 aa, synthetic construct
EVQLLESGGGLVQPGGSLRLSCAASGRTFSKHAMGWFRQAPGKGLEFVSAIDWSGGSTYYADSVKGRFTISRDNSKNTVYLQMNSLRAEDTAVYYCAADSYTDYAQLWLPELESEYDYWGQGTLVTVSS

SEQ ID NO: 17 - protein, 131 aa, synthetic construct
QVQLVESGGGLVQPGGSLRLSCAASGRTFSSYTMGWFRQAPGKGREFVSAISWSAGRTYYADSVKGRFTISRDNAKNTVYLQMNALRAEDTAVYYCAAEEAPDWAPIDCSGYGCLSLYDYWGQGTLVTVSS

SEQ ID NO: 18 - protein, 5 aa, synthetic construct
RDTMG

SEQ ID NO: 19 - protein, 16 aa, synthetic construct
VISSSGNTNYADSVLG

SEQ ID NO: 20 - protein, 8 aa, synthetic construct
HRTYGVDY

SEQ ID NO: 21 - protein, 6 aa, synthetic construct
SHNVMG

SEQ ID NO: 22 - protein, 16 aa, synthetic construct
SIGSGGSTNYVDSVKG

SEQ ID NO: 23 - protein, 8 aa, synthetic construct
VVGVYRGS

SEQ ID NO: 24 - protein, 116 aa, synthetic construct
EVQLVESGGGLIQPGGSLRLSCAASGSISSRDTMGWYRQAPGKGREMVSVISSSGNTNYADSVLGRFTISRDNAKNTVYLQMNALRAEDTAVYKCYAHRTYGVDYWGQGTLVTVSS

SEQ ID NO: 25 - protein, 117 aa, synthetic construct
QLQLLESGGGLVQPGGSLRLSCAASRSITFSHNVMGWYRQAPGKGRELVSSIGSGGSTNYVDSVKGRFTISRDNSKKTLYLQMNSLRAEDTAVYYCGVVVGVYRGSLGQGTLVTVSS

SEQ ID NO: 26 - protein, 350 aa, synthetic construct
EVQLLESGGGLVQPGGSLRLSCAASGRTFSKHAMGWFRQAPGKGLEFVSAIDWSGGSTYYADSVKGRFTISRDNSKNTVYLQMNSLRAEDTAVYYCAADSYTDYAQLWLPELESEYDYWGQGTLVTVSSCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 27 - protein, 338 aa, synthetic construct
QLQLLESGGGLVQAGGSLRLSCAASRSITFSHNVMGWYRQAPGKGRELVSSIGSGGSTNYVDSVKGRFTISRDNSKKTLYLQMNSLRAEDTAVYYCGVVVGVYRGSLGQGTLVTVSSCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 337 aa, synthetic construct
EVQLVESGGGLIQPGGSLRLSCAASGSISSRDTMGWYRQAPGKGREMVSVISSSGNTNYADSVLGRFTISRDNAKNTVYLQMNALRAEDTAVYKCYAHRTYGVDYWGQGTLVTVSSCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 476 aa, synthetic construct
EVQLVESGGGLIQPGGSLRLSCAASGSISSRDTMGWYRQAPGKGREMVSVISSSGNTNYADSVLGRFTISRDNAKNTVYLQMNALRAEDTAVYKCYAHRTYGVDYWGQGTLVTVSSGGGSQVQLVESGGGLVQPGGSLRLSCAASGRTFSSYTMGWFRQAPGKGREFVSAISWSAGRTYYADSVKGRFTISRDNAKNTVYLQMNALRAEDTAVYYCAAEEAPDWAPIDCSGYGCLSLYDYWGQGTLVTVSDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 30 - protein, 226 aa, synthetic construct
DKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 338 aa, synthetic construct
QLQLLESGGGLVQPGGSLRLSCAASRSITFSHNVMGWYRQAPGKGRELVSSIGSGGSTNYVDSVKGRFTISRDNSKKTLYLQMNSLRAEDTAVYYCGVVVGVYRGSLGQGTLVTVSSCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 117 aa, synthetic construct
QLQLLESGGGLVQAGGSLRLSCAASRSITFSHNVMGWYRQAPGKGRELVSSIGSGGSTNYVDSVKGRFTISRDNSKKTLYLQMNSLRAEDTAVYYCGVVVGVYRGSLGQGTLVTVSS

SEQ ID NO: 33 - protein, 5 aa, synthetic construct
SCDKT

SEQ ID NO: 34 - protein, 6 aa, synthetic construct
KSCDKT

(19) United States
(12) Patent Application Publication
(10) Pub. No.: US 2025/0179197 A1
(43) Pub. Date: Jun. 5, 2025

(54) HETEROMERIC AGONISTIC ANTIBODIES TO IL-18 RECEPTOR

(71) Applicant: DIAGONAL THERAPEUTICS INC., Cambridge, MA (US)

(72) Inventors: Alexey Alexandrovich Lugovskoy, Belmont, MA (US); Jean-Christophe Hus, Concord, MA (US); Melissa Geddie, Arlington, MA (US)

(21) Appl. No.: 18/923,323

(22) Filed: Oct. 22, 2024

Related U.S. Application Data
(60) Provisional application No. 63/545,278, filed on Oct. 23, 2023.

(51) Int. Cl. C07K 16/28; A61K 39/00; A61P 37/06

(52) U.S. Cl. CPC C07K 16/2866; A61P 37/06; A61K 2039/505; C07K 2317/24; C07K 2317/31; C07K 2317/53; C07K 2317/569; C07K 2317/624; C07K 2317/71; C07K 2317/75; C07K 2317/92

(57) ABSTRACT

Provided herein are bispecific agonist antibodies specific for IL-18 receptor, and methods and compositions comprising said antibodies.

### RELATED APPLICATIONS

This application claims priority to U.S. Provisional Patent Application Ser. No. 63/545,278, filed Oct. 23, 2023, the entire disclosure of which is hereby incorporated herein by reference.

### SEQUENCE LISTING

The instant application contains a Sequence Listing which has been submitted electronically in XML format and is hereby incorporated by reference in its entirety. Said XML file, created on Feb. 5, 2025, is named 758420_DGT9-001CIP_ST26.xml and is 33,124 bytes in size.

### BACKGROUND

Interleukin-18 (IL-18) (also known as interferon-gamma inducing factor, IGIF) is a pleiotropic proinflammatory cytokine that modulates both the innate and adaptive immune system responses. It has been established that IL-18 plays an important role in modulating in the inflammatory cascade, which makes it an ideal target for inhibition in autoimmune diseases, including but not limited to inflammatory diseases of the bowel, heart, and lung (Kaplanski G. Immunol. Rev. (2018); 281 (1): 138-153).

The IL-18 receptor (IL-18R) complex is a heterodimeric receptor (IL-18Rα/IL-Rβ) that is expressed on a variety of cells, including macrophages, neutrophils, natural killer (NK) cells, and antigen experienced T cells (Gracie J. A., et al. J. Leukoc. Biol. (2003); 73 (2): 213-24). Upon binding of IL-18 to the IL-18Rα subunit, IL-18Rβ is recruited to form a high-affinity complex-inducing signaling pathways shared with other IL-1R family members. These downstream signaling effector pathways are shared with other critical immune regulatory molecules such as Toll-like receptors cells (Gracie J. A., et al. J. Leukoc. Biol. (2003); 73 (2): 213-24).

While the approach of administering a therapeutic agent comprising of only a recombinant or engineered IL-18 is being investigated in early clinical trials, there are several factors that can lead to the failure of such treatment. For example, IL-18 alone binds to the extracellular signaling domain component, IL-18Rα, but does not bind to the adaptor molecule of the IL-18R receptor complex, IL-18Rβ (Takei S. et al; Arthritis Res. Ther. (2011); 13 (2): R52). However, binding to both subunits of the IL-18R is required for signal transduction and, therefore, activation of inflammatory mediators. The recombinant IL-18 is rapidly neutralized by its endogenous inhibitor IL-18 binding protein (IL-18BP) that is induced by IL-18R signaling. While IL-18 can be engineered not to bind to IL-18BP (Zhao T et al; Nature; 583, 609-14 (2020)), the mutein molecule carries a significant mutation load that makes it potentially immunogenic and therefore prone to rapid neutralization by the host immune system. Recombinant or endogenous IL-18 is a high affinity, short half-life protein that gets trapped locally at the site of administration and is eliminated quickly, which limits its ability to induce signaling in tumors distal to the administration site. Therefore, there remains a need for a stable agonist of IL-18R that binds both subunits with high affinity.

### SUMMARY

In one aspect, provided herein is a multispecific binding protein comprising at least a first binding moiety which binds specifically to human interleukin-18 receptor α (IL-18Rα) and at least a second binding moiety which binds specifically to human interleukin-18 receptor β (IL-18RB), wherein the first binding moiety comprises an amino acid sequence of SEQ ID NO: 16 or SEQ ID NO: 17; and the second binding moiety comprises an amino acid sequence of SEQ ID NO: 24, SEQ ID NO: 25, or SEQ ID NO: 32.

In some embodiments, the IL-18Rα binding moiety comprises (a) a HCDR1 sequence comprising the amino acid sequence of KHAMG (SEQ ID NO: 10), a HCDR2 sequence comprising the amino acid sequence of AIDWSGGSTYYADSVKG (SEQ ID NO: 11), a HCDR3 sequence comprising the amino acid sequence of DSYTDYAQLWLPELESEYDY (SEQ ID NO: 12); or (b) a HCDR1 sequence comprising the amino acid sequence of SYTMG (SEQ ID NO: 13), a HCDR2 sequence comprising the amino acid sequence of AISWSAGRTYYADSVKG (SEQ ID NO: 14), a HCDR3 sequence comprising the amino acid sequence of EEAPDWAPIDCSGYGCLSLYDY (SEQ ID NO: 15).

In some embodiments, the IL-18Rβ binding moiety comprises (a) a HCDR1 sequence comprising the amino acid sequence of SHNVMG (SEQ ID NO: 21), a HCDR2 sequence comprising the amino acid sequence of SIGSGGSTNYVDSVKG (SEQ ID NO: 22), a HCDR3 sequence comprising the amino acid sequence of VVGVYRGS (SEQ ID NO: 23); or (b) a HCDR1 sequence comprising the amino acid sequence of RDTMG (SEQ ID NO: 18), a HCDR2 sequence comprising the amino acid sequence of VISSSGNTNYADSVLG (SEQ ID NO: 19), a HCDR3 sequence comprising the amino acid sequence of HRTYGVDY (SEQ ID NO: 20).

In some embodiments, the first binding moiety and the second binding moiety are VHH domains. In some embodiments, the first and the second binding moieties are on the same polypeptide. In some embodiments, the first and the second binding moieties are on different polypeptides.

In some embodiments, the first and/or the second binding moiety further comprises an amino acid substitution at position 14 from the N-terminus of the binding moiety. In some embodiments, the amino acid at position 14 from the N-terminus of the binding moiety is a proline (P). In some embodiments, the substitution at position 14 from the N-terminus of the binding moiety comprises an alanine (A). In some embodiments, the substitution at position 14 from the N-terminus of the binding moiety is an alanine (A). In some embodiments, the substitution at position 14 from the N-terminus of the binding moiety further stabilizes the binding moiety. In some embodiments, the substitution at position 14 from the N-terminus of the binding moiety increases the agonist properties of the binding moiety.

In some embodiments, the multispecific binding protein further comprises one or more modified hinge regions.

In some embodiments, the one or more hinge regions comprise: an upper hinge region of up to 7 amino acids in length or is absent; and a middle hinge region and a lower hinge region, wherein the lower hinge region is linked to the N-terminus of a heavy chain constant region. In some embodiments, the upper hinge region of the first and the second modified hinge regions are the same sequence. In some embodiments, the upper hinge region of the first and the second modified hinge regions are different sequences.

In some embodiments, the upper hinge region comprises an amino acid sequence derived from an upper hinge region of a human IgG antibody. In some embodiments, the IgG antibody is selected from IgG1, IgG2, IgG3, and IgG4. In some embodiments, the IgG antibody is IgG1. In some embodiments, the upper hinge region comprises an amino acid sequence of SEQ ID NO: 2. In some embodiments, the upper hinge region comprises an amino acid sequence of SEQ ID NO: 5. In some embodiments, the IgG antibody is IgG4. In some embodiments, the upper hinge region comprises an amino acid sequence of SEQ ID NO: 4. In some embodiments, the upper hinge is absent.

In some embodiments, the multispecific binding protein further comprises all or part of an immunoglobulin Fc domain or variant thereof. In some embodiments, the Fc domain or variant thereof comprises a first Fc heavy chain and a second Fc heavy chain.

In some embodiments, the multispecific binding protein further comprises a variant Fc domain with reduced effector function. In some embodiments, at least one Fc heavy chain comprises a substitution at amino acid position 234, according to EU numbering. In some embodiments, the substitution at amino acid position 234 is an alanine (A). In some embodiments, at least one Fc heavy chain comprises a substitution at amino acid position 235, according to EU numbering. In some embodiments, the substitution at amino acid position 235 is an alanine (A). In some embodiments, at least one Fc heavy chain comprises a substitution at amino acid position 237, according to EU numbering. In some embodiments, the substitution at amino acid position 237 is an alanine (A).

In some embodiments, at least one Fc heavy chain comprises one or more substitutions at amino acid positions 234, 235, or 237, according to EU numbering. In some embodiments, the substitution at amino acid position 234 is an alanine (A), the substitution at amino acid position 235 is an alanine (A), and the substitution at amino acid position 237 is an alanine (A).

In some embodiments, the Fc domain comprises heterodimerization mutations to promote heterodimerization of the first binding moiety with the second binding moiety. In some embodiments, the heterodimerization mutations are Knob-in-Hole (KIH) mutations. In some embodiments, the first Fc heavy chain comprises an amino acid substitution at position 366, 368, or 407 which produced a hole, and the second Fc heavy chain comprises an amino acid substitution at position 366 which produce a knob. In some embodiments, the first Fc heavy chain comprises the amino acid substitution T366S, L368A, or Y407V, and the second Fc heavy chain comprises the amino acid substitution T366W.

In some embodiments, the heterodimerization mutations are charge stabilization mutations. In some embodiments, the first Fc heavy chain comprises the amino acid substitution N297K, and the second Fc heavy chain comprises the amino acid substitution N297D.

In some embodiments, the first Fc heavy chain comprises the amino acid substitution T299K, and the second Fc heavy chain comprises the amino acid substitution T299D.

In some embodiments, the heterodimerization mutations comprise an engineered disulfide bond. In some embodiments, the engineered disulfide bond is formed by a first Fc heavy chain comprising the amino acid substitution Y349C, and a second Fc heavy chain comprising the amino acid substitution S354C. In some embodiments, the engineered disulfide bond is formed by a C-terminal extension peptide fused to the C-terminus of each of the first Fc heavy chain and the second Fc heavy chain. In some embodiments, the first Fc heavy chain C-terminal extension comprises the amino acid sequence GEC, and the second Fc heavy chain C-terminal extension comprises the amino acid sequence SCDKT (SEQ ID NO: 33).

In some embodiments, at least one Fc domain comprises one or more mutations to promote increased half-life. In some embodiments, at least one Fc heavy chain comprises one or more substitutions at amino acid positions 252, 254, or 256, according to EU numbering. In some embodiments, the substitution at amino acid position 252 is a tyrosine (Y), the substitution at amino acid position 254 is a threonine (T), and the substitution at amino acid position 236 is a glutamic acid (E).

In some embodiments, the first binding moiety which binds specifically to human IL-18Rα comprises an amino acid sequence set forth in SEQ ID NOs: 26, and the second binding moiety which binds specifically to human IL-18Rβ comprises an amino acid sequence set for the in any one of SEQ ID NOs: 27 or 28 or 31.

In some embodiments, the binding protein comprises an amino acid sequence set forth in SEQ ID NO: 29. In some embodiments, the binding protein further comprises an Fc region comprising the amino acid sequence set forth in SEQ ID NO: 30.

In some embodiments, the multispecific binding protein comprises a first polypeptide chain comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to the amino acid sequence of SEQ ID NO: 26, and a second polypeptide chain comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to the amino acid sequence of SEQ ID NO: 28.

In some embodiments, the multispecific binding protein comprises a first polypeptide chain consisting of the amino acid sequence of SEQ ID NO: 26, and a second polypeptide chain consisting of the amino acid sequence of SEQ ID NO: 28.

In some embodiments, the multispecific binding protein comprises a first polypeptide chain comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to the amino acid sequence of SEQ ID NO: 29, and a second polypeptide chain comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to the amino acid sequence of SEQ ID NO: 30.

In some embodiments, the multispecific binding protein comprises a first polypeptide chain consisting of the amino acid sequence of SEQ ID NO: 29, and a second polypeptide chain consisting of the amino acid sequence of SEQ ID NO: 30.

In some embodiments, the multispecific binding protein comprises a first polypeptide chain comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to the amino acid sequence of SEQ ID NO: 26, and a second polypeptide chain comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to the amino acid sequence of SEQ ID NO: 27.

In some embodiments, the multispecific binding protein comprises a first polypeptide chain consisting of the amino acid sequence of SEQ ID NO: 26, and a second polypeptide chain consisting of the amino acid sequence of SEQ ID NO: 27.

In some embodiments, the multispecific binding protein comprises a first polypeptide chain comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to the amino acid sequence of SEQ ID NO: 26, and a second polypeptide chain comprising at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to the amino acid sequence of SEQ ID NO: 31.

In some embodiments, the multispecific binding protein comprises a first polypeptide chain consisting of the amino acid sequence of SEQ ID NO: 26, and a second polypeptide chain consisting of the amino acid sequence of SEQ ID NO: 31.

In an aspect, provided herein is a pharmaceutical composition comprising the multi-specific binding protein of any one of the preceding claims and a pharmaceutically acceptable carrier.

In another aspect, provided herein is an isolated nucleic acid molecule encoding the multispecific binding protein as described herein.

In an aspect, provided herein is an expression vector comprising a nucleic acid as described herein.

In another aspect, provided herein is a host cell comprising an expression vector as described herein.

In one aspect, the disclosure provides a multi-specific binding protein comprising a first binding moiety which binds specifically to human IL-18Rα and a second binding moiety which binds specifically to human IL-18RB, wherein the multispecific binding protein stimulates anti-tumor cytokine production without substantially stimulating MCP-1 production, GM-CSF production, or production of markers of acute inflammatory or Th2 response relative to IL-18 stimulation of MCP-1 production, GM-CSF production or production of markers of acute inflammatory or Th2 response.

In certain embodiments, the anti-tumor cytokines are selected from the group consisting of IFN-gamma, IL-2, IL-12, IL-15, CD40L, and TNFα.

In certain embodiments, the markers of acute inflammatory or Th2 response are selected from the group consisting of IL-6, IL-1B, IL-8, IL-4, IL-5, and IL-13.

In certain embodiments, the anti-tumor cytokines and markers of acute inflammatory or Th2 response are determined in a peripheral blood mononuclear cell (PBMC) assay.

In certain embodiments, the PBMC assay comprises: 1) incubating a first PBMC population with the multi-specific binding protein for at least 24 hours (e.g., 24 hours, 36 hours, 48 hours, 60 hours, 72 hours, 84 hours, or 96 hours); 2) incubating a second PBMC population with IL-18 for at least 24 hours (e.g., 24 hours, 36 hours, 48 hours, 60 hours, 72 hours, 84 hours, or 96 hours); and 3) measuring production of the anti-tumor cytokines and markers of acute inflammatory or Th2 response from the first and second PBMC population.

In certain embodiments, the multispecific binding protein stimulates production of markers of acute inflammatory or Th2 response at least 5-fold less, at least 10-fold less, at least 50-fold less, or at least 100-fold less than IL-18.

In certain embodiments, the multispecific binding protein stimulates production of markers of acute inflammatory or Th2 response at least 5-fold less, at least 10-fold less, at least 50-fold less, or at least 100-fold less than IL-18, as measured in the PBMC assay.

In certain embodiments, the multispecific binding protein stimulates production of GM-CSF at least 5-fold less, at least 10-fold less, at least 50-fold less, or at least 100-fold less than IFN-gamma production.

In an aspect, provided herein is a method for treating a disease or disorder in a subject by administering to the subject a multi-specific binding protein comprising a means for specifically binding to human IL-18Rα and a means for specifically binding to human IL-18RB.

In certain aspects, the disclosure provides a method for inducing agonist activity of IL-18 receptor signaling in a subject, the method comprising administering to the subject a multi-specific binding protein comprising a first binding moiety which binds specifically to human IL-18Rα and a second binding moiety which binds specifically to human IL-18RB, wherein the multispecific binding protein is capable of inducing IL-18 receptor signaling by inducing proximity between the IL-18Rα and IL-18Rβ subunits of human IL-18R.

In certain aspects, the disclosure provides a method for inducing agonist activity of IL-18 receptor signaling in a subject, the method comprising administering to the subject a multi-specific binding protein comprising a means for specifically binding to human IL-18Rα and a means for specifically binding to human IL-18RB.

In one aspect, the disclosure provides a method of stimulating IL-18R-mediated IFN-gamma expression in a subject, the method comprising administering to the subject a multi-specific binding protein comprising a first binding moiety which binds specifically to human IL-18Rα and a second binding moiety which binds specifically to human IL-18Rβ, wherein the multispecific binding protein stimulates anti-tumor cytokine production in the subject without substantially stimulating MCP-1 production, GM-CSF production, or production of markers of acute inflammatory or Th2 response in the subject relative to IL-18 stimulation of MCP-1 production, GM-CSF production, or production of markers of acute inflammatory or Th2 response.

In certain embodiments, the anti-tumor cytokines are selected from the group consisting of IFN-gamma, IL-2, IL-12, IL-15, CD40L, and TNFα.

In certain embodiments, the markers of acute inflammatory or Th2 response are selected from the group consisting of IL-6, IL-1B, IL-8, IL-4, IL-5, and IL-13.

In certain embodiments, the multispecific binding protein stimulates production of markers of acute inflammatory or Th2 response at least 5-fold less, at least 10-fold less, at least 50-fold less, or at least 100-fold less than IL-18.

In certain embodiments, the multispecific binding protein stimulates production of GM-CSF in the subject at least 5-fold less, at least 10-fold less, at least 50-fold less, or at least 100-fold less than IFN-gamma production in the subject.

In an aspect, the disclosure provides a method for treating a disease or disorder in a subject, comprising administering to a subject in need thereof the multispecific binding protein described herein.

In an aspect, the disclosure provides a method of stimulating IL-18R-mediated IFN-gamma expression in a subject, the method comprising administering to the multispecific binding protein described herein.

In some embodiments, the multispecific binding protein stimulates anti-tumor cytokine production in the subject without substantially stimulating MCP-1 production, GM-CSF production, or production of markers of acute inflammatory or Th2 response in the subject relative to IL-18 stimulation of MCP-1 production, GM-CSF production, or production of markers of acute inflammatory or Th2 response.

In some embodiments, the anti-tumor cytokines are selected from the group consisting of IFN-gamma, IL-2, IL-12, IL-15, CD40L, and TNFα.

In some embodiments, the markers of acute inflammatory or Th2 response are selected from the group consisting of IL-6, IL-1B, IL-8, IL-4, IL-5, and IL-13.

In some embodiments, the multispecific binding protein stimulates production of markers of acute inflammatory or Th2 response at least 5-fold less, at least 10-fold less, at least 50-fold less, or at least 100-fold less than IL-18.

In some embodiments, the multispecific binding protein stimulates production of GM-CSF in the subject at least 5-fold less, at least 10-fold less, at least 50-fold less, or at least 100-fold less than IFN-gamma production in the subject.

### BRIEF DESCRIPTION OF THE DRAWINGS

FIG. 1 is a graph illustrating agonism of DGL207, DGL333, and DGL620 in a HEK Blue assay.

FIG. 2A-2H illustrate the effects of agonistic antibodies on various immune cell markers when cultured in the presence of PBMCs. Depicted are IFN-γ (FIG. 2A), IL-5 (FIG. 2B), IL-1β (FIG. 2C), MCP-1 (FIG. 2D), IL-6 (FIG. 2E), IL-13 (FIG. 2F), GM-CSF (FIG. 2G), and IL-4 (FIG. 2H).

FIG. 3 is gene expression data from PBMC samples cultured with IL-18R agonistic antibodies. Data demonstrates that genes associated with IFNγ signaling, anti-viral responses, NK and T cell activation and lymphocyte signaling increase with the antibodies, while neutrophil activation, monocyte activation, and proinflammatory signatures decrease.

FIG. 4A and FIG. 4B depict the measurement of IFNγ and CD8 T cells in a graft vs. host disease (GvHD) mouse model.

FIG. 5A-5C demonstrate CD69 expression on CD8 T cells over 21 days for DGL336 (FIG. 5A), DGL346 (FIG. 5B), and DGL620 (FIG. 5C).

FIG. 6A-6C demonstrate a decrease in CD159a receptor on CD56+NK cells over 21 days for DGL336 (FIG. 6A), DGL346 (FIG. 6B), and DGL620 (FIG. 6C).

FIG. 7A-7C demonstrate IFNγ expression over 21 days for DGL336 (FIG. 7A), DGL346 (FIG. 7B), and DGL620 (FIG. 7C).

FIG. 8A-8C illustrate IL-2 expression over 21 days for DGL336 (FIG. 8A), DGL346 (FIG. 8B) and DGL620 (FIG. 8C).

FIG. 9A-9C illustrate monocyte numbers after exposure to DGL336 (FIG. 9A), DGL346 (FIG. 9B), or DGL620 (FIG. 9C) over 21 days.

FIG. 10A-10C illustrate IL-6 expression over 21 days for DGL336 (FIG. 10A), DGL346 (FIG. 10B), and DGL620 (FIG. 10C).

FIG. 11A-11C illustrate GM-CSF expression 21 days for DGL336 (FIG. 11A), DGL346 (FIG. 11B), and DGL620 (FIG. 11C).

FIG. 12A-12C demonstrate IL-12/23 p40 expression over 21 days for DGL336 (FIG. 12A), DGL346 (FIG. 12B), and DGL620 (FIG. 12C).

FIG. 13A-13C demonstrate TNFa expression over 21 days for DGL336 (FIG. 13A), DGL346 (FIG. 13B), and DGL620 (FIG. 13C).

FIG. 14A-14C illustrate IL-15 expression over 21 days for DGL336 (FIG. 14A), DGL346 (FIG. 14B) and DGL620 (FIG. 14C).

FIG. 15A-15C illustrate sCD40L expression over 21 days for DGL336 (FIG. 15A), DGL346 (FIG. 15B), or DGL620 (FIG. 15C).

FIG. 16A-16C illustrate MCP-1 expression over 21 days for DGL336 (FIG. 16A), DGL346 (FIG. 16B), and DGL620 (FIG. 16C).

FIG. 17A-17C illustrate IL-4 expression 21 days for DGL336 (FIG. 17A), DGL346 (FIG. 17B), and DGL620 (FIG. 17C).

FIG. 18A-18C illustrate IL-5 expression over 21 days for DGL336 (FIG. 18A), DGL346 (FIG. 18B), and DGL620 (FIG. 18C).

FIG. 19A-19C illustrate IL-13 expression 21 days for DGL336 (FIG. 19A), DGL346 (FIG. 19B), and DGL620 (FIG. 19C).

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs.

Although any methods and materials similar or equivalent to those described herein can be used in the practice of the present disclosure, exemplary methods and materials are now described. All publications mentioned herein are incorporated herein by reference to describe in their entirety.

As used herein, the terms “IL-18” or “interleukin-18” refer to the pro-inflammatory cytokine that is also known as interferon-γ inducing factor (IGIF). IL-18 has various functions in addition to an ability to induce interferon gamma, including activation of NF-κB, Fas ligand expression, and induction of both CC and CXC chemokines. due to the ability of IL-18 to induce interferon-γ production in T cells and NK cells, it plays an important role in Th1-type immune responses and participates in both innate and acquired immunity. IL-18 is related to the IL-1 family in terms of both structure and function.

The biological activities of IL-18 are mediated through IL-18 binding to a heterodimeric IL-18 receptor (IL-18R) that consists of two subunits: the α-subunit (a member of the IL-1R family, also termed IL-1R-related protein-1 or IL-1Rrp1) and the β-subunit (also termed IL-18R accessory protein, IL-18AP or AcPL). The IL-18Rα subunit binds IL-18 directly but is incapable of signal transduction. The β-subunit does not bind IL-18 by itself, but in conjunction with the α-subunit forms the high affinity receptor (KD=˜0.3 nM) that is required for signal transduction (Sims, J. E., (2002) Current Opin. Immunol. 14:117-122). IL-18 signal transduction via the IL-18Rαβ complex is similar to the IL-1R and Toll like receptor (TLR) systems. IL-18R signaling uses the signal transduction molecules, such as MyD88, IRAK, TRAF6 and results in similar responses (e.g., activation of NIK, IκB kinases, NF-κB, INK and p38 MAP kinase) as does IL-1. Requirement for IL-18Rα and signal transduction molecules in mediating IL-18 bioactivity has been confirmed using IL-18Rα subunit (Hoshino K., et al (1999) J. Immunol. 162:5041-5044;), MyD88 (Adachi O., et al. (1998) Immunity 9:143-150) or IRAK (Kanakaraj P., (1999) J. Exp. Med. 189:1129-1138) knockouts respectively.

In certain exemplary embodiments, the IL-18 cytokine is human IL-18 (Uniprot Q14116-1). In certain exemplary embodiments, IL-18 receptor is the human IL-18 receptor as represented by the human IL-18Rα (Uniprot Q13478) and IL-18Rβ sequence (Uniprot 095256-1).

As used herein, the terms “interleukin-18 receptor” or “IL-18R” or “IL18 R” or “IL-18 receptor” refer to the receptor to which IL-18 binds. IL-18R is a member of the immunoglobulin superfamily. The receptor comprises two subunits: an α-subunit (a member of the IL-1R family, also termed IL-1R-related protein-1 or IL-1Rrp1) and the β-subunit (also termed IL-18R accessory protein, IL-18AP or AcPL). The IL-18Rα subunit binds IL-18 directly but is incapable of signal transduction. The β-subunit does not bind IL-18 by itself, but in conjunction with the α-subunit forms the high affinity receptor (KD=˜ 0.3 nM) that is required for signal transduction (Sims, J. E., (2002) Current Opin. Immunol. 14:117-122). In some embodiments, the IL-18R is a human IL-18R.

As used herein, the term “inducing proximity between the IL-18Rα and IL-18Rβ subunits of human IL-18R” refers to bringing the IL-18Rα and IL-18Rβ subunits together such that human IL-18R activity is stimulated. In certain embodiments, the proximity induced by the multi-specific binding proteins of the disclosure is the same or similar to the proximity induced when IL-18 brings the IL-18Rα and IL-18Rβ subunits of human IL-18R together.

As used herein, the term “antigen-binding moiety” or “binding domain” or “binding specificity” refers to a molecule that specifically binds to an antigen as such binding is understood by one skilled in the art. For example, an antigen-binding moiety that specifically binds to an antigen binds to other molecules, generally with lower affinity as determined by, e.g., immunoassays, BIAcore®, KinExA 3000 instrument (Sapidyne Instruments, Boise, ID), or other assays known in the art. In certain embodiments, an antigen-binding moiety that specifically binds to an antigen binds to the antigen with a Ka that is at least 2 logs (e.g., factors of 10), 2.5 logs, 3 logs, 4 logs or greater than the Ka when the molecule binds non-specifically to another antigen.

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies (VHH), affibodies, common light chain antibodies, Fab fragments, F(ab′) 2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof.

As used herein, the term “heavy chain” when used in reference to an antibody can refer to any distinct type, e.g., alpha (a), delta (8), epsilon (¿), gamma (Y), and mu (u), based on the amino acid sequence of the constant domain, which give rise to IgA, IgD, IgE, IgG, and IgM classes of antibodies, respectively, including subclasses of IgG, e.g., IgG1, IgG2, IgG3, and IgG4.

As used herein, the term “full-length antibody heavy chain” refers to an antibody heavy chain comprising, from N to C terminal, a VH, a CH1 region, a hinge region, a CH2 domain and a CH3 domain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain. As used herein, the term “complementarity determining region” or “CDR” refers to sequences of amino acids within antibody variable regions, which confer antigen specificity and binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M. P. et al., “IMGT unique numbering for immunoglobulin and T cell receptor variable domains and Ig superfamily V-like domains,” Dev. Comp. Immunol., 2003 January; 27 (1): 55-77 (“IMGT” numbering scheme), and Honegger A. and Pluckthun A., “Yet another numbering scheme for immunoglobulin variable domains: an automatic modeling and analysis tool,” J. Mol. Biol., 2001 Jun. 8; 309 (3): 657-70, (AHo numbering scheme).

The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

As used herein, the term “single chain variable fragment” (scFv) refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the terms “VH” and “VL” refer to antibody heavy and light chain variable domains, respectively, as described in Kabat et al., (1991) Sequences of Proteins of Immunological Interest (NIH Publication No. 91-3242, Bethesda), which is herein incorporated by reference in its entirety. As used herein, the term “VH/VL pair” refers to a combination of a VH and a VL that together form the binding site for an antigen.

As used herein, the terms “VHH domain” or “VHH antibody” refer to the heavy chain variable domain of a camelid heavy chain-only antibody (HCAb) and humanized variants thereof, as described in Hamers-Casterman C. et al., Nature (1993) 363:446-8.10.1038/363446a0, which is incorporated by reference herein in its entirety. VHH domains or VHH antibodies are also known as “single domain” antibodies.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “multispecific antigen-binding molecules,” as used herein refers to bispecific, tri-specific or multi-specific antigen-binding molecules, and antigen-binding fragments thereof. Multispecific antigen-binding molecules may be specific for different epitopes of one target polypeptide or may contain antigen-binding domains specific for epitopes of more than one target polypeptide. In certain embodiment, the multispecific antigen binding molecules of the disclosure comprises at least a first binding specificity for the IL-18Rα subunit and at least a second binding specificity for the IL-18Rβ subunit. A multispecific antigen-binding molecule can be a single multifunctional polypeptide, or it can be a multimeric complex of two or more polypeptides that are covalently or non-covalently associated with one another. The term “multispecific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multispecific antigen-binding molecules” also includes bispecific, trispecific or multispecific antibodies or antigen-binding fragments thereof. In some embodiments, an antibody of the present disclosure is functionally linked to another antibody or antigen-binding fragment thereof to produce a bispecific antibody with a second binding specificity.

Methods for making bispecific antibodies are well-known. Traditionally, the recombinant production of bispecific antibodies was based on the co-expression of two immunoglobulin heavy chain/light chain pairs, where the two heavy chains have different specificities (Milstein et al., Nature 305:537 (1983)). Because of the random assortment of immunoglobulin heavy and light chains, the hybridomas (quadromas) produce a potential mixture of ten different antibody molecules, of which only one has the correct bispecific structure. The purification of the correct molecule is usually accomplished by affinity chromatography steps. More modern techniques for generating bispecific antibodies employ heterodimerization domains that favor desired pairing of heavy chain from the antibody with a first specificity to the heavy chain of an antibody with a second specificity.

Antibody variable domains with the desired binding specificities can be fused to immunoglobulin constant domain sequences. The fusion typically is with an immunoglobulin heavy chain constant domain, comprising at least part of the hinge, CH2 and CH3 regions. It may have the first heavy chain constant region (CH1) containing the site necessary for light chain binding present in at least one of the fusions. DNA encoding the immunoglobulin heavy chain fusions and, if desired, the immunoglobulin light chain, are inserted into separate expression vectors, and are co-transformed into a suitable host organism. For further details of generating bispecific antibodies see, for example Suresh et al., Meth. Enzymol. 121:210 (1986).

As used herein, the terms “Fc region” or “Fc domain” refer to a polypeptide comprising a CH2 domain and a CH3 domain, wherein the C-terminus of the CH2 domain is linked (directly or indirectly) to the N-terminus of the CH3 domain. In some embodiments, the Fc region is a variant Fc region. In some embodiments, the variant Fc region comprises amino acid substitutions to improve stability. In some embodiments, the variant Fc region comprises amino acid substitutions to promote heterodimerization. In some embodiments, the variant Fc region comprises amino acid substitutions to reduce or eliminate antibody effector functions. The term “Fc polypeptide” includes an antibody heavy chain linked to an antibody light chain by disulfide bonds (e.g., to form a half-antibody).

As used herein, the term “CH1 domain” refers to the first constant domain of an antibody heavy chain (e.g., amino acid positions 118-215 of human IgG1, according to the EU index). The term includes naturally occurring CH1 domains and engineered variants of naturally occurring CH1 domains (e.g., CH1 domains comprising one or more amino acid insertions, deletions, substitutions, or modifications relative to a naturally occurring CH1 domain).

As used herein, the term “hinge region” refers to the portion of an antibody heavy chain comprising the cysteine residues (e.g., the cysteine residues at amino acid positions 226 and 229 of human IgG1, according to the EU index) that mediate disulfide bonding between two heavy chains in an intact antibody. The term includes naturally occurring hinge regions and engineered variants of naturally occurring hinge regions (e.g., hinge regions comprising one or more amino acid insertions, deletions, substitutions, or modifications relative to a naturally occurring hinge regions). An exemplary full-length IgG1 hinge region comprises amino acid positions 216-230 of human IgG1, according to the EU index. A hinge region may consist of at least 2 (e.g., 5, 10, 15, 20, 40, 60 or more) amino acids which result in a flexible or semi-flexible linkage between adjacent variable regions and/or constant domains in a single polypeptide molecule. In some embodiments, the immunoglobulin-like hinge region can be from or derived from any IgG1, IgG2, IgG3, or IgG4 subtype, or from IgA, IgE, IgD, or IgM, including chimeric forms thereof.

In some embodiments, the hinge region can be from the human IgG1 subtype extending from amino acid 216 to amino acid 230 according to the numbering system of the EU index, or from amino acid 226 to amino acid 243 according to the numbering system of Kabat. Those skilled in the art may differ in their understanding of the exact amino acids corresponding to the various domains of the IgG molecule. Thus, the N-terminal or C-terminal of the domains outlined above may extend or be shortened by 1, 2, 3, 4, 5, 6, 7, 8, 9, or even 10 amino acids.

The term “upper hinge” as used herein typically refers to the last residue of the CH1 domain up to but not including the first inter-heavy chain cysteine. The upper hinge can sometimes be defined as the N-terminal sequence from position 216 to position 225 according to the Kabat EU numbering system of an IgG1 antibody (Kabat et al., Sequences of Proteins of Immunological Interest, 5th Ed. Public Health Service, National Institute of Health, Bethesda, Md., 1991). The term “middle hinge” refers to the region extending from the first inter-heavy chain cysteine to a proline residue adjacent to the carboxyl-end of the last middle hinge cysteine. The middle hinge can be the N-terminal sequence from position 226 to position 230 according to the Kabat EU numbering system. The term “lower hinge” refers to a highly conserved 7-8 amino acids. The lower hinge can be defined as the sequence from position 231 to 238 according the Kabat EU numbering system of an IgG1 antibody. In some embodiments, the antibody according to the present invention effectively comprises an upper, a middle, and a lower hinge.

As used herein, the term “a modified hinge region” refers to a hinge region in which alterations are made in one or more of the characteristics of the hinge, including, but not limited to, flexibility, length, conformation, charge and hydrophobicity relative to a wild-type hinge. The modified hinge regions disclosed herein may be generated by methods well known in the art, such as, for example introducing a modification into a wild-type hinge. In some embodiments, the hinge region may be modified by one or more amino acids. Modifications which may be utilized to generate a modified hinge region include, but are not limited to, amino acid insertions, deletions, substitutions, and rearrangements. Said modifications of the hinge and the modified hinge regions disclosed are referred to herein jointly as “hinge modifications of the invention”, “modified hinge(s) of the invention” or simply “hinge modifications” or “modified hinge(s).” The modified hinge regions disclosed herein may be incorporated into a molecule of choice including, but not limited to, antibodies and fragments thereof. In some embodiments, the hinge region may be truncated and contain only a portion of the full hinge region. As demonstrated herein, molecules comprising a modified hinge may exhibit altered (e.g., enhanced) agonistic activity when compared to a molecule having the same amino acid sequence except for the modified hinge, such as, for example, a molecule having the same amino acid sequence except comprising a wild type hinge. In some embodiments, the antibody comprises a modified hinge region wherein the upper hinge region is up to 7 amino acids in length. In some embodiments, the upper hinge region is absent. In some embodiments, the modified hinge is a modified IgG1 linker. In some embodiments, the modified IgG1 hinge is derived from the sequence PLAPDKTHT (SEQ ID NO: 1). In some embodiments, the modified IgG1 hinge comprises the sequence PLAP (SEQ ID NO: 2). In some embodiments, the modified IgG1 hinge comprises the sequence DKTHT (SEQ ID NO: 5). In some embodiments, the modified hinge is a modified IgG4 hinge. In some embodiments, the modified IgG1 hinge comprises the sequence EKSYGPP (SEQ ID NO: 4). In some embodiments, the modified hinge is a Gly/Ser hinge. In some embodiments, the Gly/Ser hinge comprises the sequence GGGGSGGGGSGGGGGGGGS (SEQ ID NO: 3).

TABLE 1
Hinge sequences.

| | SEQ | |
|---|---|---|
| | ID | |
| | NO | SEQUENCE |
| Hinge 2 | 1 | PLAPDKTHT |
| Hinge 3 | 2 | PLAP |
| Hinge 4 | 3 | GGGGSGGGGS |
| | | GGGGSGGGGS |
| Hinge 5 | 4 | EKSYGPP |
| Hinge 6 | 5 | DKTHT |
| Middle and Lower Hinge | 6 | CPPCPAPELLG |
| Hinge 3 + Middle and | 7 | PLAPCPPCPA |
| Lower | | PELLG |
| Hinge 6 + Middle and | 8 | DKTHTCPPCP |
| Lower | | APELLG |
| Hinge 5 + Middle and | 9 | EKSYGPPCPP |
| Lower | | CPAPELLG |

As used herein, the term “EU index” refers to the EU numbering convention for the constant regions of an antibody, as described in Edelman, G M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. All numbering of amino acid positions of the Fc polypeptides, or fragments thereof, used herein is according to the EU index.

In some embodiments, the term “linker” refers to 1-100 contiguous amino acid residues. Typically, a linker provides flexibility and spatial separation between two amino acids or between two polypeptide domains. A linker may be inserted between VH, VL, CH and/or CL domains to provide sufficient flexibility and mobility for the domains of the light and heavy chains depending on the format of the molecule. A linker is typically inserted at the transition between variable domains between variable and knockout domain, or between variable and constant domains, respectively, at the amino sequence level. The transition between domains can be identified because the approximate sizes of the immunoglobulin domains are well understood. The precise location of a domain transition can be determined by locating peptide stretches that do not form secondary structural elements such as beta-sheets or alpha-helices as demonstrated by experimental data or as can be determined by techniques of modeling or secondary structure prediction.

As used herein, the term “specifically binds,” “specifically binding,” “binding specificity” or “specifically recognized” refers that an antigen binding protein or antigen-binding fragment thereof that exhibits appreciable affinity for an antigen (e.g., an IL-18R antigen) and does not exhibit significant cross reactivity to a target that is not an IL-18R protein. As used herein, the term “affinity” refers to the strength of the interaction between an antigen binding protein or antigen-binding fragment thereof antigen binding site and the epitope to which it binds. In certain exemplary embodiments, affinity is measured by surface plasmon resonance (SPR), e.g., in a Biacore instrument. As readily understood by those skilled in the art, an antigen binding protein affinity may be reported as a dissociation constant (KD) in molarity (M). The antigen binding protein or antigen-binding fragment thereof of the disclosure have KD values in the range of about 10-5 M to about 10-12 M (i.e., low micromolar to picomolar range), about 10-7 M to 10-11 M, about 10-8 M to about 10-10 M, about 10-9 M. In certain embodiments, the antigen binding protein or antigen-binding fragment thereof has a binding affinity of about 10-5 M, 10-6 M, 10-7 M, 10-8 M, 10-9 M, 10-10 M, 10-11 M, or 10-12 M. In certain embodiments, the antigen binding protein or antigen-binding fragment thereof has a binding affinity of about 10-7 M to about 10-9 M (nanomolar range).

Specific binding can be determined according to any art-recognized means for determining such binding. In some embodiments, specific binding is determined by competitive binding assays (e.g., ELISA) or Biacore assays. In certain embodiments, the assay is conducted at about 20° C., 25° C., 30° C., or 37° C.

As used herein, “administer” or “administration” refers to the act of injecting or otherwise physically delivering a substance as it exists outside the body (e.g., an isolated binding polypeptide provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms.

As used herein, the term “composition” is intended to encompass a product containing the specified ingredients (e.g., an isolated binding polypeptide provided herein) in, optionally, the specified amounts, as well as any product which results, directly or indirectly, from combination of the specified ingredients in, optionally, the specified amounts.

“Effective amount” means the amount of active pharmaceutical agent (e.g., an isolated binding polypeptide of the present disclosure) sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject can be a mammal, such as a non-primate (e.g., cows, pigs, horses, cats, dogs, rats, mice, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets.

As used herein, the term “therapy” refers to any protocol, method and/or agent that can be used in the prevention, management, treatment and/or amelioration of a disease or a symptom related thereto. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as an isolated binding polypeptide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

The term “about” or “approximately” means within about 20%, such as within about 10%, within about 5%, or within about 1% or less of a given value or range.

### The IL-18R Signaling Pathway

IL-18 was originally discovered as a pro-inflammatory, IFN-γ-inducing cytokine that shares biological functions and acts synergistically with IL-12. As a member of the IL-1 family of cytokines, IL-18 is thought to play a role in early inflammatory responses and is synthesized by a range of both hematopoietic and non-hematopoietic cells (e.g., macrophages, dendritic cells, Kupffer cells, keratinocytes, osteoblasts, astrocytes, adrenal cortex cells, intestinal epithelial cells, microglial cells, and synovial fibroblasts) both constitutively and in response to lipopolysaccharide and other cytokines such as TNF-α, and is post-translationally cleaved by the caspase-1 for functional activity of the mature 18 kDa species. Active IL-18 then targets cells that express the IL-18 receptor which is widely expressed on both hematopoietic and non-hematopoietic tissues. The IL-18 receptor is a heterodimeric transmembrane protein comprised of a ligand binding IL-18R alpha (IL-18Rα) subunit and a non-ligand binding IL-18R beta (IL-18Rβ) subunit that is essential for functional signaling. Ligand-induced activation of the receptor results in recruitment and activation of intracellular myeloid differentiation 88 (MyD88) and IL-1R-associated kinase (IRAK) that simultaneously triggers at least two divergent phosphorylation cascades that activate the PI3K pathway and the MAPK pathway including activation of Akt, p38 and SAPK/JNK. Activation of these pathways culminate in NF-κB activation and transcription of its downstream genes that includes IFN-γ, chemokines, transcription factors, G protein and cell surface receptors. IL-18 shares elements of its signaling pathway with IL-1 but also bears distinct elements.

IL-18 stimulation can enhance T and NK cell maturation, cytokine secretion, cytotoxicity, and adhesion. Differentiation of naive T cells induced by IL-18 can induce either Th1 or Th2 lineages independently of either IL-4 or IL-12. In differentiated Th1 clones, IL-18 can induce the production and secretion of IFN-γ, granulocyte-macrophage colony-stimulating factor (GM-CSF), or tumor necrosis factor (TNF); and it does so primarily in synergy with IL-12. In neutrophils, IL-18 has been shown to induce the expression and secretion of cytokines and chemokines, up-regulate the expression of the cell surface adhesion molecule CD11b, and potentiate the neutrophil respiratory burst. Importantly, the IL-18 receptor itself can be up-regulated on naive T, Th1 and B cells by IL-12 which explains, in part, the synergy between these two cytokines. IL-18 also acts synergistically with IL-2 inducing expression of IL-13 (in an IFN-γ-dependent manner) and IL-10 (in an IFN-γ independent manner). Together, these results underscore the role of IL-18 in both innate and adaptive immune responses.

In non-hematopoietic cells such as endothelial and epithelial cells, synovial fibroblasts and chondrocytes, IL-18 can up-regulate the expression of adhesion molecules (such as E-selectin, ICAM and VCAM), other cytokines, chemokines (CXCL8, CXCL5, CXCL1, CXCL12, CCL, CCL20) and angiogenic mediators such as vascular endothelial growth factor (VEGF) and thrombospondin. Overall, the effects of IL-18 induction of these effectors are to increase leukocyte recruitment, cellular adhesion, extravasation of immune cells, and promotion of cellular migration and formation of new blood vessels.

In the context of numerous inflammatory diseases, IL-18 has been shown to be upregulated, to correlate with disease or to be a risk factor for disease development. Examples include in Crohn's disease, rheumatoid arthritis, systemic lupus erythrites, cardiovascular disease. Increased IL-18 levels have been observed in individuals at risk of developing either Type I (TID) or Type 2 diabetes (T2D). Elevated IL-18 has also been observed in the serum, urine, and islets of juvenile and adult T1D and T2D patients, correlating with the severity of disease, and the development of sequelae such as diabetic nephropathy. Studies on Alzheimer's patients have revealed expression of IL-18 is increased in the brain and is thought to contribute to immune and inflammatory processes that enhance oxidative stress and alter the expression of proteins that contribute to amyloid beta (Aβ) formation.

Together, these studies suggest that inflammatory disorders may represent a class of pathologies that blockade of anti-IL-18-mediated signals through the use of antagonizing anti-IL-18 may show efficacy and for which there are ample opportunities for clearly defined pre-clinical study. However, the role of agonistic antibodies in upregulating the IL-18 signaling pathway has not yet been explored. IL-18 is a potent immunostimulatory cytokine that selectively activates tumor infiltrating NK and antigen-experienced T cells, but it induces its own inhibitor IL-18 binding protein that acts as an immune checkpoint (Dinarello C. A. et al., Front. Immunol. 2013, Zhou T. et al., Nature 2020). This biologically embedded constraint on the proinflammatory signal limits the utility of IL-18 as an oncology agent. A heteromeric antibody acting as an agonist of IL-18 receptor bypasses this regulatory mechanism and can promote sustained proinflammatory signaling in tumors activating cytotoxic tumor infiltrating lymphocytes. Importantly, such an antibody will act through NFκB signaling cascade working orthogonally and synergistically with γc cytokine family (e.g., IL-2, IL-15) and IL-12 that act through JAK/TYK/STAT signaling pathway.

### Anti-IL-18Rα Binding Domains

In some embodiments, the multispecific binding protein as described herein comprises a binding moiety, binding domain, or binding specificity which binds the IL-18Rα subunit of the IL-18R (e.g., human IL-18R). Any type of binding moiety that specifically binds the IL-18Rα receptor subunit can be employed in the multispecific binding proteins disclosed herein. In certain embodiments, the binding moiety comprises an antibody variable domain. Exemplary binding moieties comprising an antibody variable domain include, without limitation, a VH, a VL, a VHH, a VH/VL pair, an scFv, a diabody, or a Fab. Other suitable binding moiety formats include, without limitation, lipocalins (see e.g., Gebauer M. et al., 2012, Method Enzymol. 503:157-188, which is incorporated by reference herein in its entirety), adnectins (see e.g., Lipovsek D., 2011, Protein Eng. Des. Sel. 24:3-9, which is incorporated by reference herein in its entirety), avimers (see e.g., Silverman J, et al., 2005, Nat. Biotechnol. 23:1556-1561, which is incorporated by reference herein in its entirety), fynomers (see e.g., Schlatter D, et al., 2012, mAbs 4:497-508, which is incorporated by reference herein in its entirety), kunitz domains (see e.g., Hosse R. J. et al., 2006, Protein Sci. 15:14-27, which is incorporated by reference herein in its entirety), knottins (see e.g., Kintzing J. R. et al., 2016, Curr. Opin. Chem. Biol. 34:143-150, which is incorporated by reference herein in its entirety), affibodies (see e.g., Feldwisch J. et al., 2010 J. Mol. Biol. 398:232-247, which is incorporated by reference herein in its entirety), and DARPins (see e.g., Pluckthun A., 2015, Annu. Rev. Pharmacol. Toxicol. 55:489-511, which is incorporated by reference herein in its entirety).

In some embodiments, the binding domain comprises the heavy and/or light chain variable regions of a conventional antibody or antigen binding fragment thereof (e.g., a Fab or scFv), wherein the term “conventional antibody” is used herein to describe heterotetrameric antibodies containing heavy and light immunoglobulin chains arranged according to the “Y” configuration. Such conventional antibodies may derive from any suitable species including but not limited to antibodies of llama, alpaca, camel, mouse, rat, rabbit, goat, hamster, chicken, monkey, or human origin. In certain exemplary embodiments, the conventional antibody comprises a heavy chain variable domain (VH) and a light chain variable domain (VL) wherein the VH and/or VL domains or one or more complementarity determining regions (CDRs) thereof are derived from the same antibodies. In certain embodiments, the conventional antibody antigen binding region may be referred to as a “Fab” (Fragment antigen-binding). The Fab comprises one constant and one variable domain from each of heavy chain and light chain. The variable heavy and light chains contain the CDRs responsible for antigen binding.

In some embodiments, the IL-18Rα receptor subunit binding subunit comprises at least a CDR or VHH domain of a VHH antibody or Nanobody. VHH antibodies, which are camelid-derived heavy chain antibodies, are composed of two heavy chains and are devoid of light chains (Hamers-Casterman, et al. Nature. 1993; 363; 446-8). Each heavy chain of the VHH antibody has a variable domain at the N-terminus, and these variable domains are referred to in the art as “VHH” domains in order to distinguish them from the variable domains of the heavy chains of the conventional antibodies i.e., the VH domains. Similar to conventional antibodies, the VHH domains of the molecule comprise HCDR1, HCDR2 and HCDR3 regions which confer antigen binding specificity and therefore VHH antibodies or fragments such as isolated VHH domains, are suitable as components of the multispecific binding proteins of the present disclosure.

Exemplary VHH CDRs or VHH domains with specificity for IL-18Rα are provided in Table 2 and Table 3 below, respectively.

TABLE 2
IL-18Rα VHH heavy chain CDR regions.

| Clone | HCDR1 | HCDR2 | HCDR3 |
|---|---|---|---|
| name | (Kabat) | (Kabat) | (Kabat) |
| VHH285_ | KHAMG | AIDWSGGST | DSYTDYA |
| alpha | (SEQ ID | YYADSVKG | QLWLPEL |
| | NO: 10) | (SEQ ID | ESEYDY |
| | | NO: 11) | (SEQ ID |
| | | | NO: 12) |
| VHH363_ | SYTMG | AISWSAGR | EEAPDWAPIDC |
| alpha | (SEQ ID | TYYADSVKG | SGYGCLSLYDY |
| | NO: 13) | (SEQ ID | (SEQ ID |
| | | NO: 14) | NO: 15) |

TABLE 3
IL-18Rα VHH domain sequences.

| Clone | |
|---|---|
| name | VHH sequence |
| VHH285_ | EVQLLESGGGLVQPGGSLRLSCAASGR |
| alpha_ | TFSKHAMGWFRQAPGKGLEFVSAIDWS |
| V2A | GGSTYYADSVKGRFTISRDNSKNTVYL |
| | QMNSLRAEDTAVYYCAADSYTDYAQL |
| | WLPELESEYDYWGQGTLVTVSS |
| | (SEQ ID NO: 16) |
| VHH363_ | QVQLVESGGGLVQPGGSLRLSCAASGR |
| alpha_ | TFSSYTMGWFRQAPGKGREFVSAISWS |
| hu1 | AGRTYYADSVKGRFTISRDNAKNTVYL |
| | QMNALRAEDTAVYYCAAEEAPDWAPID |
| | CSGYGCLSLYDYWGQGTLVTVSS |
| | (SEQ ID NO: 17) |

In some embodiments, the IL-18Rα binding domain comprises an amino acid sequence that is at least 75% identical (e.g., at least 75, 76, 77, 78, 79, 80, 81, 82, 83, 84, 85, 86, 87, 88, 89, 90, 91, 92, 93, 94, 95, 96, 97, 98, 99, or 100% identical) to at least one of the amino acid sequence of Table 2 or Table 3.

### Anti-IL-18Rβ Binding Domains

In some embodiments, the multispecific binding protein comprises a binding domain or binding specificity directed to the IL-18Rβ subunit of the IL-18R (e.g., human IL-18R).

In some embodiments, the binding domain comprises the heavy and/or light chain variable regions of a conventional antibody or antigen binding fragment thereof. In some embodiments, the binding domain is a Fab or scFv. In some embodiments, IL-18Rβ binding domain is a Fab or scFv and is paired with an IL-18Rα binding domain that is a Fab or scFV. In some embodiments, the IL-18Rβ binding domain is a Fab that shares a common light chain with a Fab of the IL-18Rα binding domain.

Exemplary binding moieties comprising an antibody variable domain include, without limitation, a VH, a VL, a VHH, a VH/VL pair, an scFv, a diabody, or a Fab. Other suitable binding moiety formats, include, without limitation, lipocalins (see e.g., Gebauer M. et al., 2012, Method Enzymol. 503:157-188, which is incorporated by reference herein in its entirety), adnectins (see e.g., Lipovsek D., 2011, Protein Eng. Des. Sel. 24:3-9, which is incorporated by reference herein in its entirety), avimers (see e.g., Silverman J, et al., 2005, Nat. Biotechnol. 23:1556-1561, which is incorporated by reference herein in its entirety), fynomers (see e.g., Schlatter D, et al., 2012, mAbs 4:497-508, which is incorporated by reference herein in its entirety), kunitz domains (see e.g., Hosse R. J. et al., 2006, Protein Sci. 15:14-27, which is incorporated by reference herein in its entirety), knottins (see e.g., Kintzing J. R. et al., 2016, Curr. Opin. Chem. Biol. 34:143-150, which is incorporated by reference herein in its entirety), affibodies (see e.g., Feldwisch J. et al., 2010 J. Mol. Biol. 398:232-247, which is incorporated by reference herein in its entirety), and DARPins (see e.g., Pluckthun A., 2015, Annu. Rev. Pharmacol. Toxicol. 55:489-511, which is incorporated by reference herein in its entirety).

In some embodiments, the IL-18Rβ receptor subunit binding domain comprises at least a CDR or VHH domain of a VHH antibody or Nanobody. In some embodiments, the IL-18Rβ VHH binding subunit is paired with a Fab or scFv IL-18Rα binding domain. In some embodiments, the IL-18Rβ VHH binding domain is paired with an IL-18Rα VHH binding domain.

Exemplary VHH CDRs and VHH domains with specificity for IL-18Rβ are provided in Tables 4 and 5 below, respectively.

TABLE 4
IL-18RB VHH heavy chain CDR regions.

| Clone | HCDR1 | HCDR2 | HCDR3 |
|---|---|---|---|
| name | (Kabat) | (Kabat) | (Kabat) |
| VHH438_ | RDTMG | VISSSGN | HRTYGVDY |
| beta | (SEQ | TNYADSV | (SEQ ID |
| | ID NO: | LG | NO: 20) |
| | 18) | (SEQ ID | |
| | | NO: 19) | |
| VHH505_ | SHNVMG | SIGSGGS | VVGVYRGS |
| beta | (SEQ ID | TNYVDSV | (SEQ ID |
| | NO: 21) | KG | NO: 23) |
| | | (SEQ ID | |
| | | NO: 22) | |

TABLE 5
IL-18RB VHH domain sequences.

| Clone name | VHH sequence |
|---|---|
| VHH438_ | EVQLVESGGGLIQPGGSLRL |
| beta_hu1 | SCAASGSISSRDTMGWYRQA |
| | PGKGREMVSVISSSGNTNYA |
| | DSVLGRFTISRDNAKNTVYL |
| | QMNALRAEDTAVYKCYAHRT |
| | YGVDYWGQGTLVTVSS |
| | (SEQ ID NO: 24) |
| VHH_505 | QLQLLESGGGLVQPGGSLRL |
| beta_v3A | SCAASRSITFSHNVMGWYRQ |
| | APGKGRELVSSIGSGGSTNY |
| | VDSVKGRFTISRDNSKKTLY |
| | LQMNSLRAEDTAVYYCGVVV |
| | GVYRGSLGQGTLV |
| | TVSS (SEQ ID NO: 25) |
| VHH_505 | QLQLLESGGGLVQAGGSLR |
| beta_V3A | LSCAASRSITFSHNVMGWY |
| P14A | RQAPGKGRELVSSIGSGGS |
| | TNYVDSVKGRFTISRDNSK |
| | KTLYLQMNSLRAEDTAVYY |
| | CGVVVGVYRGSLGQGTLVT |
| | VSS (SEQ ID NO: 32) |

### Multispecific IL-18R Binding Proteins

In some embodiments, the IL-18Rα and IL-18Rβ binding domains disclosed herein can be paired together or operatively linked to generate a multi-specific binding protein which is capable of cross-linking the IL-18Rα and IL-18Rβ subunits of the IL-18 receptor (e.g., the human IL-18 receptor). In some embodiments, the IL-18Rα binding domain (e.g., VHH) is operatively linked (directly or indirectly) to the N and/or C terminus of a first Fc domain or polypeptide, and the IL-18RB binding domain is operatively linked to the N and/or C terminus of second Fc domain or polypeptide, such that the first Fc domain and the second Fc domain facilitate heterodimerization of the IL-18Rα and IL-18Rβ binding domains.

In some embodiments, the multispecific binding protein as disclosed herein are agonistic to the IL-18R signaling pathway, i.e., they are not antagonistic to the IL-18R pathway. In some embodiments, agonism may be measured using an IL-18 potency assay (e.g., HEK-Blue™ IL-18 potency assay (InVivogen)). In this assay, HEK-Blue™ IL-18 cells are generated by stably transfecting HEK293-derived cells with the genes encoding IL-18Rα and IL-18Rβ. In some embodiments, the responses to human TNF-α and IL-1B are blocked, which enables the cells to respond specifically to IL-18.

The HEK-Blue™ IL-18 cells also express an NF-κB/AP-1-inducible secreted embryonic alkaline phosphatase (SEAP) reporter gene. The binding of bispecific antibodies to the heterodimeric IL-18 receptor on the surface of these cells triggers a signaling cascade leading to the activation of NF-κB and the subsequent production of SEAP which can be quantified.

The multi-specific binding proteins of the disclosure possess agonist activity toward the IL-18R, stimulating IFN-gamma expression while minimally inducing IL-5 and IL-1-beta expression. This is in contrast to the natural ligand of IL-18R, IL-18, which potently stimulates IL-5 and IL-1-beta expression. Thus, the multi-specific binding proteins of the disclosure provide a uniquely specific agonism of IL-18R not found in IL-18.

Accordingly, in one aspect the disclosure provides a multi-specific binding protein comprising a first binding moiety which binds specifically to human IL-18Rα and a second binding moiety which binds specifically to human IL-18Rβ, wherein the multispecific binding protein stimulates anti-tumor cytokine production without substantially stimulating MCP-1 production, GM-CSF production, or production of markers of acute inflammatory or Th2 response relative to IL-18 stimulation of MCP-1 production, GM-CSF production or production of markers of acute inflammatory or Th2 response.

In certain embodiments, the anti-tumor cytokines are selected from the group consisting of IFN-gamma, IL-2, IL-12, IL-15, CD40L, and TNFα.

In certain embodiments, the markers of acute inflammatory or Th2 response are selected from the group consisting of IL-6, IL-1B, IL-8, IL-4, IL-5, and IL-13.

In certain embodiments, the anti-tumor cytokines and markers of acute inflammatory or Th2 response are determined in a peripheral blood mononuclear cell (PBMC) assay.

In certain embodiments, the PBMC assay comprises: 1) incubating a first PBMC population with the multi-specific binding protein for at least 24 hours (e.g., 24 hours, 36 hours, 48 hours, 60 hours, 72 hours, 84 hours, or 96 hours); 2) incubating a second PBMC population with IL-18 for at least 24 hours (e.g., 24 hours, 36 hours, 48 hours, 60 hours, 72 hours, 84 hours, or 96 hours); and 3) measuring production of the anti-tumor cytokines and markers of acute inflammatory or Th2 response from the first and second PBMC population.

In certain embodiments, between about 10,000 and about 1,000,000 PBMCs are used in the first and second PBMC populations. In certain embodiments, between about 250,000 PBMCs are used in the first and second PBMC populations.

In certain embodiments, the first PBMC population is incubated with IL-12 prior to or simultaneously with the multi-specific binding protein. In certain embodiments, the second PBMC population is incubated with IL-12 prior to or simultaneously with the IL-18. In certain embodiments, the first and second PBMC populations are incubated with IL-12 at a concentration of about 0.1 ng/ml to about 1 ng/mL. In certain embodiments, the first and second PBMC populations are incubated with IL-12 at a concentration of about 0.5 ng/ml.

In certain embodiments, the first PBMC population is incubated the multi-specific binding protein at a concentration of about 1 nM to about 1,000 nM. In certain embodiments, the first PBMC population is incubated the multi-specific binding protein at a concentration of about 300 nM.

In certain embodiments, the multispecific binding protein stimulates production of markers of acute inflammatory or Th2 response at least 5-fold less, at least 10-fold less, at least 50-fold less, or at least 100-fold less than IL-18.

In certain embodiments, the multispecific binding protein stimulates production of markers of acute inflammatory or Th2 response at least 5-fold less, at least 10-fold less, at least 50-fold less, or at least 100-fold less than IL-18, as measured in the PBMC assay.

In certain embodiments, the multispecific binding protein stimulates production of GM-CSF at least 5-fold less, at least 10-fold less, at least 50-fold less, or at least 100-fold less than IFN-gamma production.

In certain embodiments, the multispecific binding protein stimulates IL-5 production at least 5-fold less than IL-18. In certain embodiments, the multispecific binding protein stimulates IL-5 production at least 10-fold less than IL-18. In certain embodiments, the multispecific binding protein stimulates IL-5 production at least 50-fold less than IL-18. In certain embodiments, the multispecific binding protein stimulates IL-5 production at least 100-fold less than IL-18. In certain embodiments, the multispecific binding protein stimulates IL-5 production at least 500-fold less than IL-18. In certain embodiments, the multispecific binding protein stimulates IL-5 production at least 1,000-fold less than IL-18. In certain embodiments, the multispecific binding protein stimulates IL-5 production about 5-fold less to about 1,000-fold less than IL-18.

In certain embodiments, the multispecific binding protein stimulates IL-1-beta production at least 5-fold less than IL-18. In certain embodiments, the multispecific binding protein stimulates IL-1-beta production at least 10-fold less than IL-18. In certain embodiments, the multispecific binding protein stimulates IL-1-beta production at least 50-fold less than IL-18. In certain embodiments, the multispecific binding protein stimulates IL-1-beta production at least 100-fold less than IL-18. In certain embodiments, the multispecific binding protein stimulates IL-1-beta production at least 500-fold less than IL-18. In certain embodiments, the multispecific binding protein stimulates IL-1-beta production at least 1,000-fold less than IL-18. In certain embodiments, the multispecific binding protein stimulates IL-1-beta production about 5-fold less to about 1,000-fold less than IL-18.

In certain embodiments, the fold change in production of markers of acute inflammatory or Th2 response of the multispecific binding protein compared to IL-18 is as measured in the PBMC assay described herein.

In some embodiments, the multispecific binding protein comprises at least one binding domain on each polypeptide, e.g., a VHH or a Fab. In some embodiments, the multispecific binding protein comprises a tandem binding domain bispecific construct, e.g., at least two VHH domains on one polypeptide. In some embodiments, the VHH domains target two different targets, e.g., IL-18Rα and IL-18Rβ. In some embodiments, one or more of the binding domains are optimized. Optimization may include changes in the primary amino acid sequence to reduce presumably unfavorable properties (e.g., immunogenicity in a human host; humanization).

In some embodiments, the agonist activity of the multispecific binding protein that meets or exceeds a particular threshold over background when measured with an agonist activity assay, e.g., HEK-Blue assay. In some embodiments, the agonist activity of the multispecific binding protein is about 2-fold over background. In some embodiments the agonist activity of the multispecific binding protein is about 3-fold over background. In some embodiments, the agonist activity of the multispecific binding protein is about 4-fold over background. In some embodiments, the agonist activity of the multispecific binding protein is about 5-fold over background. In some embodiments, the agonist activity of the multispecific binding protein is about 6-fold over background. In some embodiments, the agonist activity of the multispecific binding protein is about 7-fold over background. In some embodiments, the agonist activity of the multispecific binding protein is about 8-fold over background. In some embodiments, the agonist activity of the multispecific binding protein is about 9-fold over background. In some embodiments the agonist activity of the multispecific binding protein is about 10-fold over background. In some embodiments, the agonist activity of the multispecific binding protein is about 11-fold over background. In some embodiments, the agonist activity of the multispecific binding protein is about 12-fold over background. In some embodiments, the agonist activity of the multispecific binding protein is about 13-fold over background. In some embodiments, the agonist activity of the multispecific binding protein is about 14-fold over background. In some embodiments, the multispecific binding protein agonist activity is about 15-fold over background. In some embodiments, the multispecific binding protein agonist activity is about 20-fold over background. In some embodiments, the agonist activity of the multispecific binding protein is about 30-fold over background. In some embodiments, the agonist activity of the multispecific binding protein is about 40-fold over background. In some embodiments, the agonist activity of the multispecific binding protein is about 50-fold over background. In some embodiments, the agonist activity of the multispecific binding protein is about 60-fold over background. In some embodiments, the agonist activity of the multispecific binding protein is about 70-fold over background. In some embodiments, the agonist activity of the multispecific binding protein is about 80-fold over background. In some embodiments, the agonist activity of the multispecific binding protein is about 90-fold over background. In some embodiments, the agonist activity of the multispecific binding protein is about 100-fold over background.

The Fc polypeptides employed in the multispecific binding proteins of the disclosure generally comprise a CH2 domain and a CH3 domain, wherein the C-terminus of the CH2 domain is linked (directly or indirectly) to the N-terminus of the CH3 domain. Any naturally occurring or variant CH2 and/or CH3 domain can be used. For example, in some embodiments, the CH2 and/or CH3 domain is a naturally occurring CH2 or CH3 domain from an IgG1, IgG2, IgG3, IgG4, IgA1, or IgA2 antibody heavy chain, e.g., a human IgG1, IgG2, IgG3, IgG4, IgA1, or IgA2 antibody heavy chain. The CH2 and CH3 domains can be from the same or different antibody heavy chains. In certain embodiments, the Fc polypeptide comprises a CH2 and CH3 domain-containing portion from a single antibody heavy chain. In certain embodiments, the CH2 and/or CH3 domain is a variant of a naturally occurring CH2 or CH3 domain, respectively. In certain embodiments, the CH2 and/or CH3 domain is a variant comprising one or more amino acid insertions, deletion, substitutions, or modifications relative to a naturally occurring CH2 or CH3 domain, respectively. In certain embodiments, the CH2 and/or CH3 domain is a chimera of one or more CH2 or CH3 domains, respectively. In certain embodiments, the CH2 domain comprises amino acid positions 231-340 of a naturally occurring hinge region (e.g., human IgG1), according to the EU index. In certain embodiments, the CH3 domain comprises amino acid positions 341-447 of a naturally occurring hinge region (e.g., human IgG1), according to the EU index.

In some embodiments, the Fc polypeptides further comprise a hinge region, wherein the C-terminus of hinge region is linked (directly or indirectly) to the N-terminus of the CH2 domain. For example, in certain embodiments, the hinge region is a naturally occurring hinge region from an IgG1, IgG2, IgG3, IgG4, IgA1, or IgA2 antibody heavy chain, e.g., a human IgG1, IgG2, IgG3, IgG4, IgA1, or IgA2 antibody heavy chain. The hinge region can be from the same or different antibody heavy chain than the CH2 and/or CH3 domains. In some embodiments, the hinge region is a variant comprising one or more amino acid insertions, deletion, substitutions, or modifications relative to a naturally occurring hinge region. In some embodiments, the hinge region is a chimera of one or more hinge regions. In some embodiments, the hinge region comprises amino acid positions 226-229 of a naturally occurring hinge region (e.g., human IgG1), according to the EU index. In some embodiments, the hinge region comprises amino acid positions 216-230 of a naturally occurring hinge region (e.g., human IgG1), according to the EU index. In some embodiments, the hinge region comprises amino acid positions 216-230 of a naturally occurring hinge region (e.g., human IgG1), according to the EU index. In some embodiments, the hinge region is a variant IgG4 hinge region comprising a serine(S) at amino acid position 228, according to the EU index.

In some embodiments, the Fc polypeptides further comprise a CH1 domain, wherein the C-terminus of CH1 domain is linked (directly or indirectly) to the N-terminus of the hinge region. For example, in some embodiments, the CH1 domain is a naturally occurring CH1 domain from an IgG1, IgG2, IgG3, IgG4, IgA1, or IgA2 antibody heavy chain, e.g., a human IgG1, IgG2, IgG3, IgG4, IgA1, or IgA2 antibody heavy chain. The CH1 domain can be from the same or different antibody heavy chain than the hinge region, CH2 domain and/or CH3 domain. In some embodiments, the CH1 domain is a variant comprising one or more amino acid insertions, deletions, substitutions, or modifications relative to a naturally occurring CH1 domain. In some embodiments, the CH1 domain is a chimera of one or more CH1 domain. In some embodiments, the CH1 domain comprises amino acid positions 118-215 of a naturally occurring hinge region (e.g., human IgG1), according to the EU index.

In some embodiments, the Fc polypeptide lacks a CH1 domain or comprises mutations in a CH1 domain or heavy chain variable domain that prevent association of the heavy chain with an antibody light chain. In some embodiments, the antibody heavy chain lacks a portion of a hinge region.

### Heterodimerization Motifs

In some embodiments, the first and second Fc domains are further engineered to enhance heterodimerization of the IL-18Rα and IL-18Rβ binding domains and minimize the effects of incorrect chain pairing (i.e., pairing of IL-18Rα binding domains or identical IL-18Rβ domains).

Any art-recognized approach that addresses the problem of incorrect chain pairing can be employed to improve desired multi-specific antibody production. For instance, US2010/0254989 A1 describes the construction of bispecific cMet-ErbB1 antibodies, where the VH and VL of the individual antibodies are fused genetically via a GlySer linker. For bispecific antibodies including an Fc domain, mutations may be introduced into the Fc to promote the correct heterodimerization of the Fc portion. Several such approaches are reviewed in Klein et al. (mAbs (2012) 4:6, 1-11), the contents of which are incorporated herein by reference in their entirety.

In some embodiments, the IL-18Rα and IL-18Rβ binding specificities of the multispecific binding protein are heterodimerized through knobs-into-holes (KiH) pairing of Fc domains. This dimerization technique utilizes “protuberances” or “knobs” with “cavities” or “holes” engineered into the interface of CH3 domains. Where a suitably positioned and dimensioned knob or hole exists at the interface of either the first or second CH3 domain, it is only necessary to engineer a corresponding hole or knob, respectively, at the adjacent interface, thus promoting and strengthening Fc domain pairing in the CH3/CH3 domain interface. The IgG Fc domain that is fused to the VHH is provided with a knob, and the IgG Fc domain of the conventional antibody is provided with a hole designed to accommodate the knob, or vice-versa. A “knob” refers to an at least one amino acid side chain, typically a larger side chain, that protrudes from the interface of the CH3 portion of a first Fc domain. The protrusion creates a “knob” which is complementary to and received by a “hole” in the CH3 portion of a second Fc domain. The “hole” is an at least one amino acid side chain, typically a smaller side chain, which recedes from the interface of the CH3 portion of the second Fc domain. This technology is described, for example, in U.S. Pat. Nos. 5,821,333; 5,731,168 and 8,216,805; Ridgway et al. Protein Engineering (1996) 9:617-621); and Carter P. J. Immunol. Methods (2001) 248:7-15, which are herein incorporated by reference.

Exemplary amino acid residues that may act as the knob include arginine (R), phenylalanine (F), tyrosine (Y) or tryptophan (W). An existing amino acid residue in the CH3 domain may be replaced or substituted with a knob amino acid residue. Preferred amino acids to substitute may include any amino acids with a small side chain, such as alanine (A), asparagine (N), aspartic acid (D), glycine (G), serine(S), threonine (T), or valine (V).

Exemplary amino acid residues that may act as the hole include alanine (A), serine (S), threonine (T), or valine (V). An existing amino acid residue in the CH3 domain may be replaced or substituted with a hole amino acid residue. Preferred amino acids to substitute may include any amino acids with a large side chain, such as arginine (R), phenylalanine (F), tyrosine (Y) or tryptophan (W).

The CH3 domain is preferably derived from a human IgG1 antibody. Exemplary amino acid substitutions to the CH3 domain include Y349C, S354C, T366S, T366Y, T366W, F405A, F405W, Y407T, Y407A, Y407V, T394S, or combinations thereof. A preferred exemplary combination is S354C, T366Y or T366W for the knob mutation on a first CH3 domain and Y349C, T366S, L368A, Y407T or Y407V for the hole mutation on a second CH3 domain.

In some embodiments, the two Fc domains of the antigen binding construct are heterodimerized through Fab arm exchange (FAE). A human IgG1 possessing a P228S hinge mutation may contain an F405L or K409R CH3 domain mutation. Mixing of the two antibodies with a reducing agent leads to FAE. This technology is described in U.S. Pat. No. 9,212,230 and Labrijn A. F. PNAS (2013) 110 (13): 5145-5150, which are incorporated herein by reference.

In some embodiments, the two Fc domains of the antigen binding construct are heterodimerized through electrostatic steering effects. This dimerization technique utilizes electrostatic steering to promote and strengthen Fc domain pairing in the CH3/CH3 domain interface. The charge complementarity between two CH3 domains is altered to favor heterodimerization (opposite charge paring) over homodimerization (same charge pairing). In this method, the electrostatic repulsive forces prevent homodimerization. Certain exemplary amino acid residue substitutions which confer electrostatic steering effects include K409D, K392D, and/or K370D in a first CH3 domain and D399K, E356K, and/or E357K in a second CH3 domain. This technology is described in US Patent Publication No. 2014/0154254 A1 and Gunasekaran K. JBC (2010) 285 (25): 19637-19646, which are incorporated herein by reference.

In some embodiments, the charge complementarity is formed by a first Fc domain comprising a N297K and/or a T299K mutation, and a second Fc domain comprising a N297D and/or a T99D mutation.

In some embodiments, the two Fc domains of the antigen binding construct are heterodimerized through hydrophobic interaction effects. This dimerization technique utilizes hydrophobic interactions instead of electrostatic ones to promote and strengthen Fc domain pairing in the CH3/CH3 domain interface. Exemplary amino acid residue substitution may include K409W, K360E, Q347E, Y349S, and/or S354C in a first CH3 domain and D399V, F405T, Q347R, E357W, and/or Y349C in a second CH3 domain. Preferred pairs of amino acid residue substitutions between a first CH3 domain and a second CH3 domain include K409W:D399V, K409W:F405T, K360E:Q347R, Y349S:E357W, and S354C:Y349C. This technology is described in US Patent Publication No. 2015/0307628 A1.

In some embodiments, heterodimerization can be mediated through the use of leucine zipper fusions. Leucine zipper domains fused to the C terminus of each CH3 domain of the antibody chains force heterodimerization. This technology is described in Wranik B. JBC (2012) 287 (52): 43331-43339.

In some embodiments, heterodimerization can be mediated through the use of a Strand Exchange Engineered Domain (SEED) body. CH3 domains derived from an IgG and IgA format force heterodimerization. This technology is described in Muda M. PEDS (2011) 24 (5): 447-454.

In some embodiments, the heterodimerization motif may comprise non-native, disulfide bonds formed by engineered cysteine residues. In certain embodiments, the first set of disulfide may comprise a Y349C mutation in the first Fc domain and a S354C mutation in the second Fc domain. In other embodiment, an engineered disulfide bond may be introduced by fusion a C-terminal extension peptide with an engineered cysteine residue to the C-terminus of each of the two Fc domains. In certain embodiments, the first Fc domain may comprise the substitution of the carboxyl-terminal as “PGK” with “GEC”, and the second Fc domain may comprise the substitution of the carboxyl terminal amino acids “PGK” with “KSCDKT (SEQ ID NO: 34)”.

In some embodiments, the multispecific binding proteins may employ the CrossMab principle (as reviewed in Klein et al.), which involves domain swapping between heavy and light chains so as to promote the formation of the correct pairings. Yet another approach involves engineering the interfaces between the paired VH-VL domains or paired CH1-CL domains of the heavy and light chains so as to increase the affinity between the heavy chain and its cognate light chain (Lewis et al. Nature Biotechnology (2014) 32:191-198).

An alternative approach to the production of multispecific binding protein preparations having the correct antigen specificity has been the development of methods that enrich for antibodies having the correct heavy chain-light chain pairings. For example, Spiess et al. (Nature Biotechnology (2013) 31:753-758) describe a method for the production of a MET-EGFR bispecific antibody from a co-culture of bacteria expressing two distinct half-antibodies.

Methods have also been described wherein the constant region of at least one of the heavy chains of a multispecific binding protein is mutated so as to alter its binding affinity for an affinity agent, for example Protein A. This allows correctly paired heavy chain heterodimers to be isolated based on a purification technique that exploits the differential binding of the two heavy chains to an affinity agent (see US2010/0331527, WO2013/136186).

International patent application no. PCT/EP2012/071866 (WO2013/064701) addresses the problem of incorrect chain pairing using a method for multispecific antibody isolation based on the use of anti-idiotypic binding agents, in particular anti-idiotypic antibodies. The anti-idiotype binding agents are employed in a two-step selection method in which a first agent is used to capture antibodies having a VH-VL domain pairing specific for a first antigen and a second agent is subsequently used to capture antibodies also having a second VH-VL domain pairing specific for a second antigen.

In some embodiments, the multispecific binding protein employs a first binding specificity having a single domain antibody (VHH) binding region and a second binding specificity comprising a VHH binding region. In some embodiments, the first and the second binding moieties are on the same polypeptide. Ins some embodiments, the first and the second binding moieties are on different polypeptides.

In some embodiments, the multispecific binding protein as described herein further comprises a common light chain. The term “common light chain” as used herein refers to a light chain which is capable of pairing with a first heavy chain of an antibody which binds to a first antigen in order to form a binding site specifically binding to said first antigen and which is also capable of pairing with a second heavy chain of an antibody which binds to a second antigen in order to form a binding site specifically binding to said second antigen. A common light chain is a polypeptide comprising in N-terminal to C-terminal direction an antibody light chain variable domain (VL), and an antibody light chain constant domain (CL), which is herein also abbreviated as “VL-CL”. Multispecific binding proteins with a common light chain require heterodimerization of the distinct heavy chains. In certain embodiments, the heterodimerization methods listed above may be used with a common light chain. In certain exemplary embodiments, the heterodimerization motif may comprise non-native, disulfide bonds formed by engineered cysteine residues. Adding disulfide bonds, both between the heavy and light chain of an antibody has been shown to improve stability. Additionally, disulfide bonds have also been used as a solution to improve light-chain pairing within bispecific antibodies (Geddie M. L. et al, mABs (2022) 14 (1)).

Unless otherwise stated, all antibody constant region numbering employed herein corresponds to the EU numbering scheme, as described in Edelman et al. (Proc. Natl. Acad. Sci. 63 (1): 78-85. 1969).

Additional methods of heterodimerization of heavy and/or light chains and the generation and purification of asymmetric antibodies are known in the art. See, for example, Klein C. mAbs (2012) 4 (6): 653-663, and U.S. Pat. No. 9,499,634, each of which is incorporated herein by reference.

### Effector Function Mutations

In some embodiments, the multispecific binding proteins as disclosed herein maybe provided as various isotypes and with different constant region variants. The Fc region of the multispecific binding primarily determines its effector function in terms of Fc binding, antibody-dependent cell-mediated cytotoxicity (ADCC) activity, complement dependent cytotoxicity (CDC) activity, and antibody-dependent cell phagocytosis (ADCP) activity. These “cellular effector functions”, as distinct from effector T cell function, involve the recruitment of cells bearing Fc receptors to the site of the target cells, resulting in killing of the antibody-bound cell.

In some embodiments, the multispecific binding protein as described herein may be one that exhibits reduced effector function. In certain embodiments, the one or more mutations reduces one or more of antibody dependent cellular cytotoxicity (ADCC), antibody dependent cellular phagocytosis (ADCP), or complement dependent cytotoxicity (CDC). In certain embodiments, a binding protein as described herein may lack ADCC, ADCP and/or CDC activity. In either case, an antibody according to the present invention may comprise, or may optionally lack, an Fc region that binds to one or more types of Fc receptor. Use of different antibody formats, and the presence or absence of FcR binding and cellular effector functions, allow the antibody to be tailored for use in particular therapeutic purposes as discussed elsewhere herein.

In some embodiments, the first and the second Fc domain comprise one or more mutations that reduces Fc effector function. In certain embodiments, the first Fc domain and the second Fc domain each comprise a L234A and L235A mutation. These IgG1 mutations are also known as the “LALA” mutations and are described in further detail in Xu et al. (Cell Immunol. 2000; 200:16-26). In certain embodiments the first Fc domain and the second Fc domain each comprise a L234A, L235A, G237A, and/or P329G mutation. The Fc domain amino acid positions referred to herein are based on EU antibody numbering. Alternatively, an antibody may have a constant region which is effector null. An antibody may have a heavy chain constant region that does not bind Fcγ receptors, for example the constant region may comprise a L235E mutation. Another optional mutation for a heavy chain constant region is S228P, which increases stability. A heavy chain constant region may be an IgG4 comprising both the L235E mutation and the S228P mutation. This “IgG4-PE” heavy chain constant region is effector null. A disabled IgG1 heavy chain constant region is also effector null. A disabled IgG1 heavy chain constant region may contain alanine at position 234, 235 and/or 237 (EU index numbering), e.g., it may be an IgG1 sequence comprising the L234A, L235A and/or G237A mutations (“LALAGA”).

Human IgG1 constant regions containing specific mutations or altered glycosylation on residue Asn297 (e.g., N297Q, N297D, and N297K, EU index numbering) have been shown to reduce binding to Fc receptors.

In other embodiments, it may be desirable to enhance the binding of the Fc region of a multispecific antibody to human Fc gamma receptor IIIA (FcgRIIIA) relative to that of the Fc region of a corresponding naturally occurring antibody. In certain embodiments, a constant region may be engineered for enhanced ADCC and/or CDC and/or ADCP. The potency of Fc-mediated effects may be enhanced by engineering the Fc domain by various established techniques. Such methods increase the affinity for certain Fc-receptors, thus creating potential diverse profiles of activation enhancement. This can be achieved by modification of one or several amino acid residues. Example mutations are one or more of the residues selected from 239, 332 and 330 for human IgG1 constant regions (or the equivalent positions in other IgG isotypes). An antibody may thus comprise a human IgG1 constant region having one or more mutations independently selected from S239D, 1332E and A330L (EU index numbering).

Increased affinity for Fc receptors can also be achieved by altering the natural glycosylation profile of the Fc domain by, for example, generating under fucosylated or de-fucosylated variants. Non-fucosylated antibodies harbor a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIA binding capacity. For example, to increase ADCC, residues in the hinge region can be altered to increase binding to FcγRIIIA. Thus, an antibody may comprise a human IgG heavy chain constant region that is a variant of a wild-type human IgG heavy chain constant region. In certain embodiments, the variant human IgG heavy chain constant region binds to human Fcγ receptors selected from the group consisting of FcγRIIB and FcγRIIA with higher affinity than the wild type human IgG heavy chain constant region binds to the human FcγRIIIA. The antibody may comprise a human IgG heavy chain constant region that is a variant of a wild type human IgG heavy chain constant region, wherein the variant human IgG heavy chain constant region binds to human FcγRIIB with higher affinity than the wild type human IgG heavy chain constant region binds to human FcγRIIB. The variant human IgG heavy chain constant region can be a variant human IgG1, a variant human IgG2, or a variant human IgG4 heavy chain constant region. In one embodiment, the variant human IgG heavy chain constant region comprises one or more amino acid mutations selected from G236D, P238D, S239D, S267E, L328F, and L328E (EU index numbering system). In another embodiment, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system).

The enhancement of CDC may be achieved by amino acid changes that increase affinity for C1q, the first component of the classic complement activation cascade. Another approach is to create a chimeric Fc domain created from human IgG1 and human IgG3 segments that exploit the higher affinity of IgG3 for C1q. Antibodies of the present invention may comprise mutated amino acids at residues 329, 331 and/or 322 to alter the C1q binding and/or reduced or abolished CDC activity. In another embodiment, the antibodies or antibody fragments disclosed herein may contain Fc regions with modifications at residues 231 and 239, whereby the amino acids are replaced to alter the ability of the antibody to fix complement. In one embodiment, the antibody or fragment has a constant region comprising one or more mutations selected from E345K, E430G, R344D and D356R, in particular a double mutation comprising R344D and D356R (EU index numbering system).

The functional properties of the multispecific binding proteins may be further tuned by combining amino acid substitutions that alter Fc binding affinity with amino acid substitutions that affect binding to FcRn. Binding proteins with amino acid substitutions that affect binding to FcRn (also referred to herein as “FcRn variants”) may in certain situations also increase serum half-life in vivo as compared to an unmodified binding protein. As will be appreciated, any combination of Fc and FcRn variants may be used to tune clearance of the antigen-antibody complex. Suitable FcRn variants that may be combined with any of the Fc variants described herein that include without limitation N434A, N434S, M428L, V308F, V259I, M428L/N434S, V259I/V308F, Y436I/M428L, Y436I/N434S, Y436V/N434S, Y436V/M428L, M252Y, M252Y/S254T/T256E, and V259I/V308F/M428L.

### Expression of Antigen-Binding Proteins

In one aspect, polynucleotides encoding the binding proteins (e.g., antigen-binding proteins and antigen-binding fragments thereof) disclosed herein are provided. Methods of making binding proteins comprising expressing these polynucleotides are also provided.

Polynucleotides encoding the binding proteins disclosed herein are typically inserted in an expression vector for introduction into host cells that may be used to produce the desired quantity of the binding proteins. Accordingly, in certain aspects, the disclosure provides expression vectors comprising polynucleotides disclosed herein and host cells comprising these vectors and polynucleotides.

The term “vector” or “expression vector” is used herein to mean vectors used in accordance with the present disclosure as a vehicle for introducing into and expressing a desired gene in a cell. As known to those skilled in the art, such vectors may readily be selected from the group consisting of plasmids, phages, viruses and retroviruses. In general, vectors compatible with the disclosure will comprise a selection marker, appropriate restriction sites to facilitate cloning of the desired gene and the ability to enter and/or replicate in eukaryotic or prokaryotic cells.

Numerous expression vector systems may be employed for the purposes of this disclosure. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the binding proteins may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding a binding protein, e.g. an antibody or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, from supernatant of lysed cells culture, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, GS-CHO and CHO-K1 (Chinese Hamster Ovary lines), DG44 and DUXB11 (Chinese Hamster Ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HEK (human kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (POTELLIGENT® cells) (Biowa, Princeton, N.J.)). In one embodiment, NSO cells may be used. CHO cells are particularly useful. Host cell lines are typically available from commercial services, e.g., the American Tissue Culture Collection, or from authors of published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the binding proteins featured in the disclosure can also be expressed in non-mammalian cells such as bacteria or yeast or plant cells. In this regard, it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the binding proteins can become part of inclusion bodies. In some embodiments, the binding proteins are then isolated, purified and assembled into functional molecules. In some embodiments, the binding proteins of the disclosure are expressed in a bacterial host cell. In some embodiments, the bacterial host cell is transformed with an expression vector comprising a nucleic acid molecule encoding a binding protein of the disclosure.

In addition to prokaryotes, eukaryotic microbes may also be used. Saccharomyces cerevisiae, or common baker's yeast, is the most commonly used among eukaryotic microbes, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### Formulations/Pharmaceutical Compositions

In certain embodiments, a pharmaceutical composition comprising a pharmaceutically acceptable carrier and a therapeutically effective amount of an antigen-binding protein described herein is provided. Some embodiments include pharmaceutical compositions comprising a therapeutically effective amount of any one of the binding proteins as described herein, or a binding protein-drug conjugate, in admixture with a pharmaceutically or physiologically acceptable formulation agent selected for suitability with the mode of administration.

Acceptable formulation materials are typically non-toxic to recipients at the dosages and concentrations employed.

In some embodiments, the pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides, e.g., sodium or potassium chloride, or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

In some embodiments the optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

In some embodiments the primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the disclosure, binding protein compositions can be prepared for storage by mixing the selected composition having the desired degree of purity with optional formulation agents in the form of a lyophilized cake or an aqueous solution. Further, the binding protein can be formulated as a lyophilizate using appropriate excipients such as sucrose.

In some embodiments, the pharmaceutical compositions of the disclosure can be selected for parenteral delivery or subcutaneous delivery. Alternatively, the compositions can be selected for inhalation or for delivery through the digestive tract, such as orally. The preparation of such pharmaceutically acceptable compositions is within the skill of the art.

In some embodiments, the formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use can be in the form of a pyrogen-free, parenterally acceptable, aqueous solution comprising the desired binding protein in a pharmaceutically acceptable vehicle. A particularly suitable vehicle for parenteral injection is sterile distilled water in which a binding protein is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a binding protein can be formulated as a dry powder for inhalation. Binding protein inhalation solutions can also be formulated with a propellant for aerosol delivery. In yet another embodiment, solutions can be nebulized.

It is also contemplated that certain formulations can be administered orally. In one embodiment of the disclosure, multispecific binding proteins that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of the binding protein. Diluents, flavorings, low melting point waxes, vegetable oils, lubricants, suspending agents, tablet disintegrating agents, and binders can also be employed.

Another pharmaceutical composition can involve an effective quantity of multi-specific binding proteins in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions of the disclosure will be evident to those skilled in the art, including formulations involving binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g., films, or microcapsules. Sustained release matrices can include polyesters, hydrogels, polylactides, copolymers of L-glutamic acid and gamma ethyl-L-glutamate, poly(2-hydroxyethyl-methacrylate), ethylene vinyl acetate, or poly-D (−)-3-hydroxybutyric acid. Sustained-release compositions can also include liposomes, which can be prepared by any of several methods known in the art.

In some embodiments, pharmaceutical compositions are to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper that can be pierced by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

The disclosure also encompasses kits for producing a single dose administration unit. The kits can each contain both a first container having a dried multispecific binding protein and a second container having an aqueous formulation. Also included within the scope of this disclosure are kits containing single and multi-chambered pre-filled syringes (e.g., liquid syringes and lyosyringes).

The effective amount of a binding protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding protein is being used, the route of administration, and the size (body weight, body surface, or organ size) and condition (the age and general health) of the patient. Accordingly, the clinician can titer the dosage and modify the route of administration to obtain the optimal therapeutic effect.

Dosing frequency will depend upon the pharmacokinetic parameters of the binding protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

In some embodiments, the composition can also be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

Multi-specific binding proteins disclosed herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma and are herein incorporated by reference in their entireties). These formulations for administration to the respiratory tract can be in the form of an aerosol or solution for a nebulizer, or as a microfine powder for insufflations, alone or in combination with an inert carrier such as lactose. In such a case, the particles of the formulation will, in one embodiment, have diameters of less than 50 microns, in one embodiment less than 10 microns.

A multi-specific binding protein disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the heterodimeric protein alone or in combination with other pharmaceutically acceptable excipients can also be administered.

Transdermal patches, including iontophoretic and electrophoretic devices, are well known to those of skill in the art, and can be used to administer a heterodimeric protein. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, all of which are herein incorporated by reference in their entireties.

In certain embodiments, a pharmaceutical composition comprising a multi-specific binding protein described herein is a lyophilized powder, which can be reconstituted for administration as solutions, emulsions and other mixtures. It may also be reconstituted and formulated as solids or gels. The lyophilized powder is prepared by dissolving heterodimeric protein described herein, or a pharmaceutically acceptable derivative thereof, in a suitable solvent. In certain embodiments, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined. Multi-specific binding proteins provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, all of which are herein incorporated by reference in their entireties. In a specific embodiment, a heterodimeric protein described herein is targeted to a tumor.

### Methods of Treatment/Use

An aspect of the disclosure is a multispecific binding protein as described herein for use as a medicament.

In some embodiments, a method of treating a disease or disorder through the activation of IL-18R is provided, the method comprising administering to a subject in need thereof an effective amount of a multispecific binding protein as described herein.

In an aspect, the disclosure provides a method for treating a disease or disorder in a subject, comprising administering to a subject in need thereof the multispecific binding protein described herein.

In an aspect, the disclosure provides a method of stimulating IL-18R-mediated IFN-gamma expression in a subject, the method comprising administering to the multispecific binding protein described herein.

In some embodiments, the multispecific binding protein stimulates anti-tumor cytokine production in the subject without substantially stimulating MCP-1 production, GM-CSF production, or production of markers of acute inflammatory or Th2 response in the subject relative to IL-18 stimulation of MCP-1 production, GM-CSF production, or production of markers of acute inflammatory or Th2 response.

In some embodiments, the anti-tumor cytokines are selected from the group consisting of IFN-gamma, IL-2, IL-12, IL-15, CD40L, and TNFα.

In some embodiments, the markers of acute inflammatory or Th2 response are selected from the group consisting of IL-6, IL-1B, IL-8, IL-4, IL-5, and IL-13.

In some embodiments, the multispecific binding protein stimulates production of markers of acute inflammatory or Th2 response at least 5-fold less, at least 10-fold less, at least 50-fold less, or at least 100-fold less than IL-18.

In some embodiments, the multispecific binding protein stimulates production of GM-CSF in the subject at least 5-fold less, at least 10-fold less, at least 50-fold less, or at least 100-fold less than IFN-gamma production in the subject.

In some embodiments, the multispecific binding protein as described herein can be employed in any known assay method, such as competitive binding assays, direct and indirect sandwich assays, and immunoprecipitation assays for the detection and quantitation of one or more target antigens. The binding proteins can bind the one or more target antigens with an affinity that is appropriate for the assay method being employed.

For diagnostic applications, in some embodiments, the multispecific binding proteins as described herein can be labeled with a detectable moiety. The detectable moiety can be any one that is capable of producing, either directly or indirectly, a detectable signal. For example, the detectable moiety can be, but not limited to, a radioisotope, such as ³H, ¹⁴C, ³²p, ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, β-galactosidase, or horseradish peroxidase.

The binding proteins as described herein can be useful for in vivo imaging. A binding protein labeled with a detectable moiety can be administered to an animal, e.g., into the bloodstream, and the presence and location of the labeled antibody in the host assayed. The binding protein can be labeled with any moiety that is detectable in an animal, whether by, but not limited to, nuclear magnetic resonance (NMR), radiology, or other detection means known in the art.

The disclosure also relates to a kit comprising a binding protein as described herein and other reagents useful for detecting target antigen levels in biological samples. Such reagents can include a detectable label, blocking serum, positive and negative control samples, and detection reagents. In some embodiments, the kit comprises a composition comprising any binding protein, polynucleotide, vector, vector system, and/or host cell as described herein.

In some embodiments, the kit comprises a container and a label or package insert on or associated with the container. Suitable containers include, for example, bottles, vials, syringes, intravenous (IV) solution bags, etc. The containers may be formed from a variety of materials such as glass or plastic. The container holds a composition which is by itself or combined with another composition effective for treating, preventing and/or diagnosing a condition and may have a sterile access port (for example the container may be an IV solution bag or a vial having a stopper that can be pierced by a hypodermic injection needle). In some embodiments, the label or package insert indicates that the composition is used for preventing, diagnosing, and/or treating the condition of choice. Alternatively, or additionally, the article of manufacture or kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the present disclosure relates to a method of preventing and/or treating a disease or disorder (e.g., cancer). In some embodiments, the method comprises administering to a patient a therapeutically effective amount of at least one of the binding proteins, or pharmaceutical compositions related thereto, described herein. In some embodiments, the patient is a human.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The following examples are put forth so as to provide those of ordinary skill in the art with a complete disclosure and description of how to make and use the methods and compositions featured in the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Humanization of IL-18R Binders

Binders previously identified with high levels of activity using the HEK Blue assay were humanized and optimized for therapeutic use. VHH binders against IL-18Rα and IL-18Rβ were modeled computationally with the antigen using the DIAGONAL Platform. Residues non-essential to epitope recognition were replaced with human sequences. Back mutations were added only to preserve antigen binding and stability. Constructs were measured for affinity using the Carterra and activity using the HEK Blue assay. Furthermore, the proline at position 14 of the VHH binding domain was substituted with an alanine in some cases to improve stability and agonism of the binder.

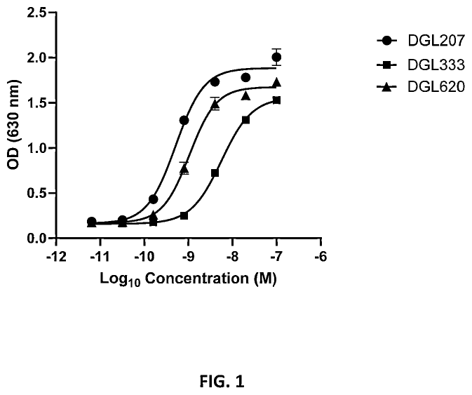

When humanizing the antibody DGL207, it was observed that the potency and affinity were reduced against IL-18Rβ (DGL333). Using the DIAGONAL platform, it was observed that the proline in position 14 (P14) could be destabilizing the molecule in the context of the rigidified hinge (hinge 1). Reverting this mutation back to alanine, which is found in the llama germline, improved both the affinity and the activity of the bispecific in the HEK Blue assay (FIG. 1).

In addition to humanizing the VHHs, the Fc was engineered to optimal therapeutic use (e.g., LALAGA, knobs in holes, and YTE mutations).

TABLE 6
Optimized IL-18R antibodies.

| | Chain 1 | Chain 2 |
|---|---|---|
| DGL336 | VHH_285a_V2A_h1 | VHH438b_hu1_h1 |
| DGL346 | VHH438b_SL_363a_ | Fc_knob |
| | hole_hu1 | |
| DGL333 | VHH_285a_V2A_h1 | VHH_505b_V3A |
| DGL620 | VHH_285a_V2A_h1 | VHH_505b_ |
| | | V3A_P14A |

TABLE 7
Sequences of optimized agonistic antibodies to IL-18R.

| Name | Sequence |
|---|---|
| VHH_285a_V2A | EVQLLESGGGLVQPGGSLRLSCAASGRTFSKHAMGWFRQAPGKG |
| h1 | LEFVSAIDWSGGSTYYADSVKGRFTISRDNSKNTVYLQMNSLRA |
| | EDTAVYYCAADSYTDYAQLWLPELESEYDYWGQGTLVTVSSCPP |
| | CPAPEAAGAPSVFLFPPKPKDTLYITREPEVTCVVVDVSHEDPE |
| | VKFNWYVDGVEVHNAKTKPREEQYNSTYRVVSVLTVLHQDWLNG |
| | KEYKCKVSNKALPAPIEKTISKAKGQPREPQVCTLPPSRDELTK |
| | NQVSLSCAVKGFYPSDIAVEWESNGQPENNYKTTPPVLDSDGSF |
| | FLVSKLTVDKSRWQQGNVFSCSVMHEALHNHYTQKSLSLSPG |
| | (SEQ ID NO: 26) |
| VHH_505b_V3A_ | QLQLLESGGGLVQAGGSLRLSCAASRSITFSHNVMGWYRQAPGK |
| P14A | GRELVSSIGSGGSTNYVDSVKGRFTISRDNSKKTLYLQMNSLRA |
| | EDTAVYYCGVVVGVYRGSLGQGTLVTVSSCPPCPAPEAAGAPSV |
| | FLFPPKPKDTLYITREPEVTCVVVDVSHEDPEVKFNWYVDGVEV |
| | HNAKTKPREEQYNSTYRVVSVLTVLHQDWLNGKEYKCKVSNKAL |
| | PAPIEKTISKAKGQPREPQVYTLPPCRDELTKNQVSLWCLVKGF |
| | YPSDIAVEWESNGQPENNYKTTPPVLDSDGSFFLYSKLTVDKSR |
| | WQQGNVFSCSVMHEALHNHYTQKSLSLSPG |
| | (SEQ ID NO: 27) |
| VHH438b_hu1_ | EVQLVESGGGLIQPGGSLRLSCAASGSISSRDTMGWYRQAPGKG |
| h1 | REMVSVISSSGNTNYADSVLGRFTISRDNAKNTVYLQMNALRAE |
| | DTAVYKCYAHRTYGVDYWGQGTLVTVSSCPPCPAPEAAGAPSVF |
| | LFPPKPKDTLYITREPEVTCVVVDVSHEDPEVKFNWYVDGVEVH |
| | NAKTKPREEQYNSTYRVVSVLTVLHQDWLNGKEYKCKVSNKALP |
| | APIEKTISKAKGQPREPQVYTLPPCRDELTKNQVSLWCLVKGFY |
| | PSDIAVEWESNGQPENNYKTTPPVLDSDGSFFLYSKLTVDKSRW |
| | QQGNVFSCSVMHEALHNHYTQKSLSLSPG (SEQ ID NO: 28) |
| VHH438b_SL_3 | EVQLVESGGGLIQPGGSLRLSCAASGSISSRDTMGWYRQAPGKG |
| 63a_hole_hu1 | REMVSVISSSGNTNYADSVLGRFTISRDNAKNTVYLQMNALRAE |
| | DTAVYKCYAHRTYGVDYWGQGTLVTVSSGGGSQVQLVESGGGLV |
| | QPGGSLRLSCAASGRTFSSYTMGWFRQAPGKGREFVSAISWSAG |
| | RTYYADSVKGRFTISRDNAKNTVYLQMNALRAEDTAVYYCAAEE |
| | APDWAPIDCSGYGCLSLYDYWGQGTLVTVSDKTHTCPPCPAPEA |
| | AGAPSVFLFPPKPKDTLYITREPEVTCVVVDVSHEDPEVKFNWY |
| | VDGVEVHNAKTKPREEQYNSTYRVVSVLTVLHQDWLNGKEYKCK |
| | VSNKALPAPIEKTISKAKGQPREPQVCTLPPSRDELTKNQVSLS |
| | CAVKGFYPSDIAVEWESNGQPENNYKTTPPVLDSDGSFFLVSKL |
| | TVDKSRWQQGNVFSCSVMHEALHNHYTQKSLSLSPG |
| | (SEQ ID NO: 29) |
| Fc_knob | DKTHTCPPCPAPEAAGAPSVFLFPPKPKDTLYITREPEVTCVVV |
| | DVSHEDPEVKFNWYVDGVEVHNAKTKPREEQYNSTYRVVSVLTV |
| | LHQDWLNGKEYKCKVSNKALPAPIEKTISKAKGQPREPQVYTLP |
| | PCRDELTKNQVSLWCLVKGFYPSDIAVEWESNGQPENNYKTTPP |
| | VLDSDGSFFLYSKLTVDKSRWQQGNVFSCSVMHEALHNHYTQKS |
| | LSLSPG (SEQ ID NO: 30) |
| VHH_505b_V3A | QLQLLESGGGLVQPGGSLRLSCAASRSITFSHNVMGWYRQAPGK |
| | GRELVSSIGSGGSTNYVDSVKGRFTISRDNSKKTLYLQMNSLRA |
| | EDTAVYYCGVVVGVYRGSLGQGTLVTVSSCPPCPAPEAAGAPSV |
| | FLFPPKPKDTLYITREPEVTCVVVDVSHEDPEVKFNWYVDGVEV |
| | HNAKTKPREEQYNSTYRVVSVLTVLHQDWLNGKEYKCKVSNKAL |
| | PAPIEKTISKAKGQPREPQVYTLPPCRDELTKNQVSLWCLVKGF |
| | YPSDIAVEWESNGQPENNYKTTPPVLDSDGSFFLYSKLTVDKSR |
| | WQQGNVFSCSVMHEALHNHYTQKSLSLSPG (SEQ ID NO: 31) |

### Example 2. Measurement of Affinity to IL-18R by Humanized Binders

SPR experiments were performed using Carterra LSA equipped with an HC30M chip (Carterra-Bio). Binding assays were carried out at 25° C. with HBSTE (10 mM HEPES ph7.4, 150 mM NaCl, 3 mM EDTA, 0.05% Tween-20) with 0.5 g/L BSA. A density of anti-human IgG-Fc capture lawn was established on an HC30M chip using amine coupling. To prepare the lawn, the chip was activated with 133 mM EDC and 33.3 mM S-NHS in 100 mM MES pH5.5. Coupling of goat anti-human IgG (H+L) multi-species SP ads-UNLB (Southern Biotech, 2087-01) with standard immobilization was done for 15 minutes in 10 mM sodium acetate pH 4.5 and quenched using 1M ethanolamine HCl pH8.5. The anti-human IgG-Fc capture surface was then used to capture a panel of antibodies at 20 μg/mL. Monomeric antigen for human IL-18Rα, human IL-18Rβ, cynomolgus IL-18Rα, or rhesus IL-18Rβ targeted by the antibodies was injected over the captured antibody array at 6 concentrations in 4-fold dilution series starting at 500 nM. Binding data was referenced and blanked from a buffer injected, then globally fit to a 1:1 Langmuir binding model for estimation of ka, kd, and KD using Carterra Kinetics Software. Table 8 shows the KD values for DGL336, DGL346, and DGL620.

TABLE 8
Measured affinities of non-humanized leads

| | | huIL18Ra |
|---|---|---|
| Antibody | Target | KD (M) |
| VHH285 | Human IL-18Rα | 1.5E−08 |
| | Cyno IL-18Rα | 1.4E−08 |
| VHH363 | Human IL-18Rα | 3.4E−08 |
| | Cyno IL-18Rα | 9.5E−08 |
| VHH436b | Human IL-18Rβ | 1.6E−09 |
| | Rhesus IL-18Rβ | 1.8E−09 |
| VHH505 | Human IL-18Rβ | 1.6E−09 |
| | Rhesus IL-18Rβ | >2E−07 |

TABLE 9
Measured affinities of leads against targets

| Antibody | Target | K_{D} (M) |
|---|---|---|
| DGL336 | Human IL- | 1.1E−08 |
| | 18Rα | |
| | Human IL- | 3.2E−09 |
| | 18Rβ | |
| | Cyno IL-18Rα | 1.4E−08 |
| | Rhesus IL- | 1.5E−08 |
| | 18Rβ | |
| DGL346 | Human IL- | 6.0E−08 |
| | 18Rα | |
| | Human IL- | 2.2E−09 |
| | 18Rβ | |
| | Cyno IL-18Rα | 9.0E−07 |
| | Rhesus IL- | 1.0E−08 |
| | 18Rβ | |
| DGL620 | Human IL- | 8.0E−09 |
| | 18Rα | |
| | Human IL- | 1.4E−08 |
| | 18Rβ | |
| | Cyno IL-18Rα | 1.0E−08 |
| | Rhesus IL- | >2E−07 |
| | 18Rβ | |

### Example 3. Evaluation of Agonistic Activity Using HEK Cells Over-Expressing IL-18Rα and IL-18Rβ

HEK Blue™ IL-18 cells were purchased from Invivogen (hbk-hmIL-18). These cell lines overexpress IL-18Rα and IL-18Rβ while blocking responses to TNFα and IL-1B. Reporter cells were revived and cultured according to supplier's recommendations. Cells were rinsed with PBS and added to 96-well plates at a density of ˜50,000 cells/well. Either controls or bispecific antibodies were added to the wells at the final agonist concentration listed in Table 10. The plate was incubated at 37° C. and 5% CO₂ for 20-24 hours. QUANTI-Blue™ solution (Invivogen) was prepared using manufacturer's instructions and 180 μL added per well to a new 96-well plate followed by the addition of 20 μL of supernatant from the antibody induced HEK-Blue IL-18 cells. The cell culture plate was incubated at 37° C. and 5% CO₂ for 3 hours and then read on a spectrophotometer (Varioskan Lux, Thermo) at 630 nm. All optimized antibodies had activity in the HEK Blue assay similar to their parental, non-humanized antibodies. To calculate EC50s, bispecifics were evaluated in a 10-point, 5-fold titration starting at 100 nM. Human IL-18 (Invivogen, rcyec-hiI18) was titrated in an 8-point, 4 fold titration starting at 1 ng/ml. All data was fitted in PRISM using the log (agonist) vs. response—variable slope (four parameters) analysis.

TABLE 10
HEK-Blue Results for optimized antibodies.

| | Abs 405 | Fold over |
|---|---|---|
| DGL | at 100 nM | background |
| DGL336 | 0.90 | 4.9 |
| DGL346 | 1.34 | 7.2 |
| DGL620 | 1.87 | 10.1 |
| PBS | 0.18 | 1.0 |
| Human IL-18 | 2.6 | 14.4 |

### Example 4. Induction of IFNγ in Human PBMCs

To assess the activity of the agonist antibodies in immune cells, frozen peripheral blood mononuclear cells (PBMCs) were obtained from six donors. These PBMCs were thawed and plated at a concentration of 250,000 cells per well in 10% RPMI (Gibco, A10491). To run the assay, IL-12 (R&D Systems, 10018-IL) was added to each well at a final concentration of 0.5 ng/mL. Bispecific antibodies were added to a final concentration at 300 nM and diluted to generate titration curves. Cells were incubated 24 hours before spinning plates at 500 g for 5 minutes and supernatants were collected. Supernatants of all samples were run on the human IFNγ DUoSet ELISA kit from R&D systems (DY285B). IL-18 (R&D Systems, 9124-IL) was used as a positive control. Plates were run on Luminex according to manufacturer instructions. Data was analyzed in PRISM using a four parameter, least squares fit. The EC50 was calculated for each bispecific antibody tested and then averaged among the six donors (Table 11). Table 12 shows data of calculated maximum induction of IFNγ after 24 hours.

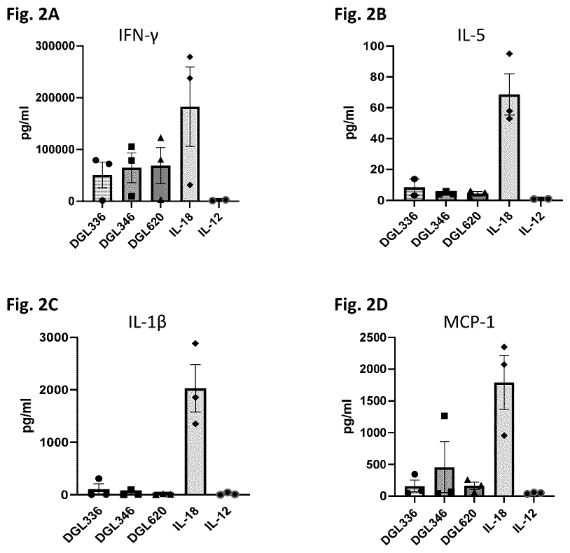

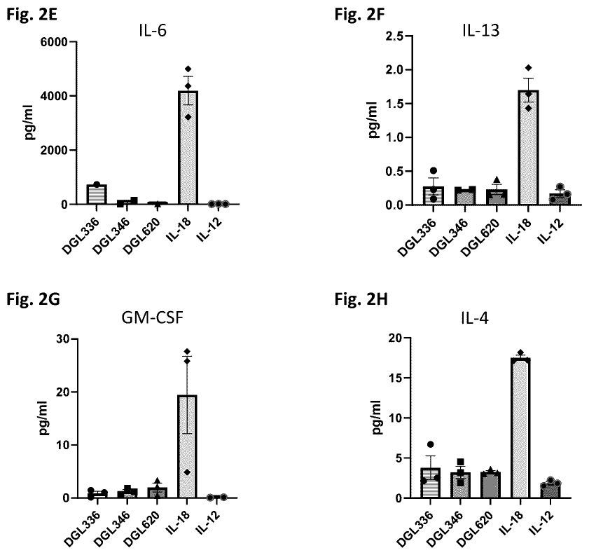

Results from the PBMC assays are shown in FIG. 2A-2H. After 24 hours, IL-18R bispecific antibodies were able to induce IFNγ, a marker of tumor effector lymphocyte activation. Unlike IL-18, which is a ligand that can activate multiple immune cells, IL-18R bispecific antibodies do not induce IL-5 and other Th2 cytokines (e.g., IL-4 and IL-13, data not shown). Further, the bispecific antibodies do not activate IL-1β and other cytokines and chemokines involved in acute inflammation such as IL-6, IL-8, MCP-1, and GM-CSF (FIG. 2B-2H). These data demonstrate that the heteromeric IL-18R bispecific agonists can preferentially stimulate anti-tumor effector lymphocytes.

TABLE 11
EC50s with different human donors.

| Donor ID | DGL336 | DGL346 | DGL620 | IL-18 |
|---|---|---|---|---|
| 1 | 0.7 | 0.6 | 0.2 | 1.9 |
| 2 | 0.5 | 1.3 | 0.3 | 1.7 |
| 3 | 1.5 | 0.3 | 0.04 | 1.4 |
| 4 | 0.3 | 0.5 | 0.1 | 4.7 |
| 5 | 0.2 | 0.1 | 0.05 | 1.9 |
| 6 | 3.6 | 2.4 | 0.2 | 29.4 |
| Average | 1.1 | 0.9 | 0.15 | 6.8 |

TABLE 12
Calculated maximum induction of IFN-γ after 24 hours

| Donor ID | DGL336 | DGL346 | DGL620 | IL-18 |
|---|---|---|---|---|
| 1 | 13000 | 16000 | 16000 | 42000 |
| 2 | 620 | 1700 | 1300 | 5300 |
| 3 | 17000 | 22000 | 22000 | 89000 |
| 4 | 9300 | 17000 | 19000 | 69000 |
| 5 | 1500 | 15000 | 15000 | 150000 |
| 6 | 1300 | 3000 | 2400 | 28000 |

### Example 5. Gene Expression Profiling in PBMCs

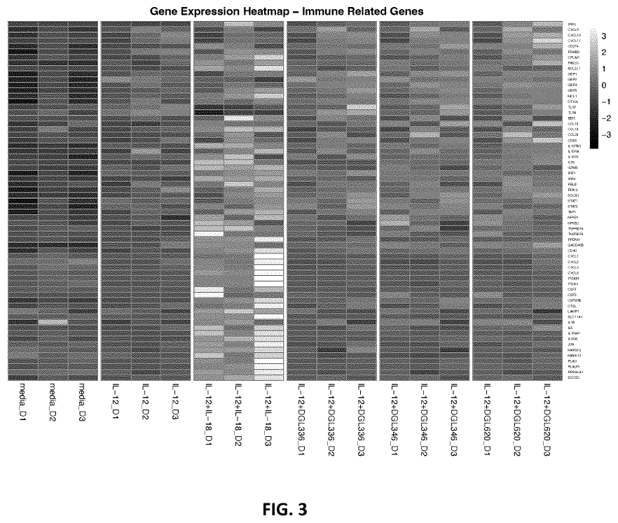

Human PBMCs were treated with recombinant human IL-12 alone (R&D Systems, 10018-IL), IL-12 plus recombinant IL-18 (rhIL-18, R&D Systems, 9124-IL), or IL-12 plus 10 nM of a DIAGONAL IL-18R antibody agonist for 24 hours. Total RNA was isolated and analyzed with the NanoString platform. All DIAGONAL IL-18R agonists tested showed selective gene expression profiles compared to rhIL-18 in human PBMCs. Specifically, the DIAGONAL IL-18R agonists induced expression of genes involved in anti-viral responses, NK and T cell activation and lymphocyte signaling. In contrast to IL-18, the IL-18R agonists did not impact genes involved in neutrophil and monocyte activation as well as genes that drive proinflammatory responses (FIG. 3).

### Example 6. In Vivo Characterization of Agonists in Murine GvHD Model

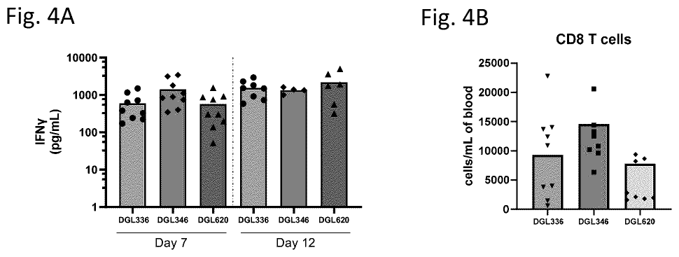

A human PBMC engrafted mouse model of graft-vs-host disease (GvHD) was used to evaluate the molecular profile of the IL-18R agonists on the immune system. Human PBMCs, isolated from healthy adult human donors, were thawed on ice from frozen stocks, washed, and resuspended in PBS at 2×10⁸ cells/mL. Seven to nine-week-old female NOD/SCID/IL-2Rγnull immunodeficient mice were engrafted with 2×10⁷ PBMCs on Day 0. Three donors were used to establish 3 cohorts of mice. Each agonist was administered at 3 mpk to a group of a group of 9 mice representing 3 mice per donor on Day 0, Day 4, and D10. Flow cytometry analysis of whole blood was used to evaluate cellularity and cell activation status while cytokine analysis was performed by CBA analysis using plasma. FIG. 4A shows the induction of IFNγ at two time points—day 7 and day 12. All agonists show a robust induction of IFNγ in the GVHD model. FIG. 4B shows the amount of CD8 T cells, which is increased after administration of the agonists.

### Example 7. In Vivo Characterization of Agonists in Cynomolgus Monkeys

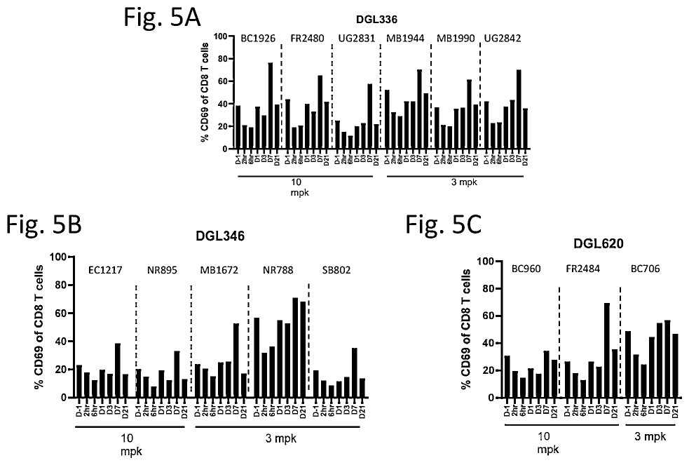

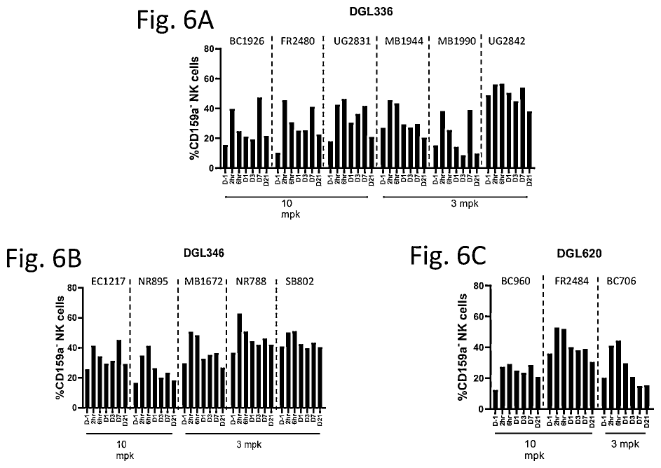

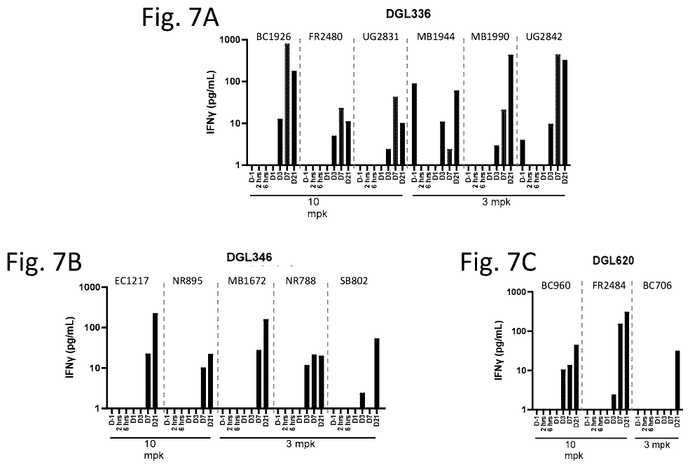

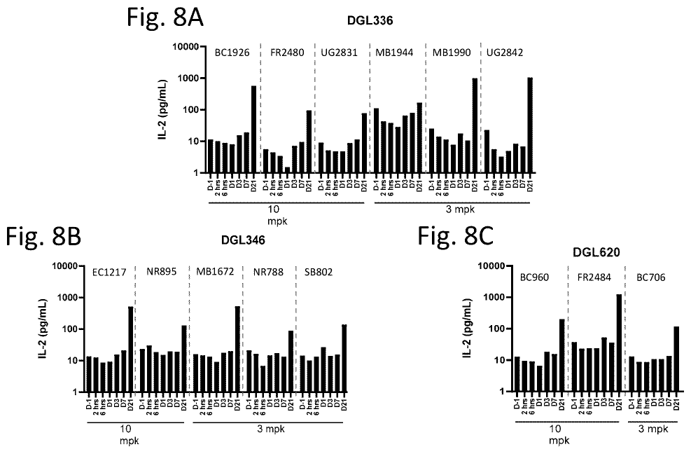

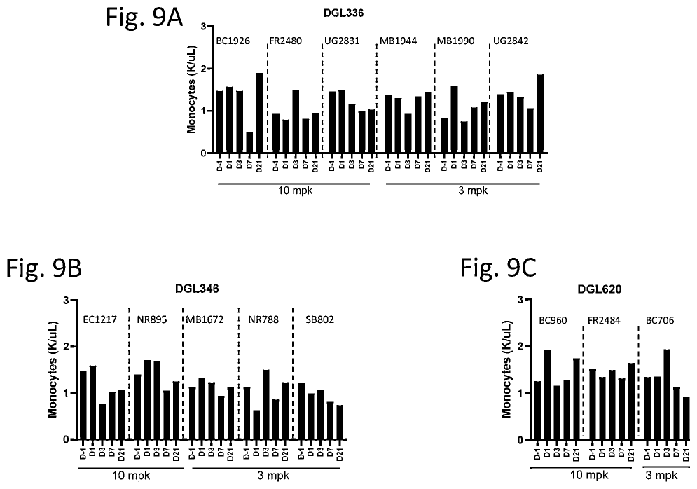

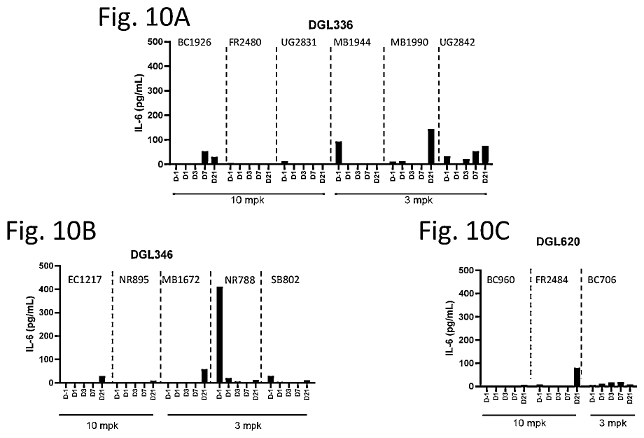

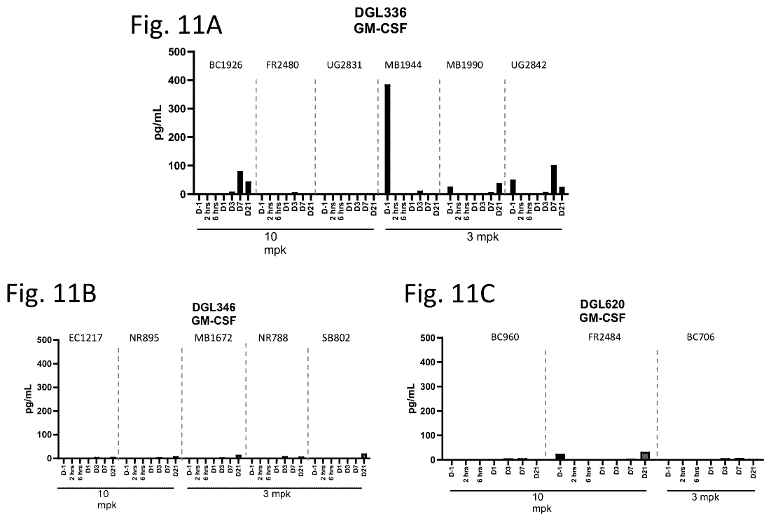

Male cynomolgus monkeys (2-3 monkeys per agonist) received a single dose of an IL-18R bispecific agonist on Day 0. Blood was collected the day prior to administration and various timepoints post dosing to evaluate the PK and PD profiles. Blood was either analyzed as fresh whole blood or processed to plasma. The data are shown in FIG. 5A-19C. The data show that a single dose of an IL-18R agonist led to both T and NK cell activation as indicated by CD69 expression on CD8 T cells (FIG. 5A-5C) and a decrease in the inhibitory CD159a receptor on CD56+ NK cells, respectively (FIG. 6A-6C). Concordant with the observed cell activation, there was an increase of the T and NK cell meditators, IFNγ (FIG. 7A-7C) and IL-2 (FIG. 8A-8C). The agonists did not expand monocytes (FIG. 9A-9C) and also did not induce acute inflammatory mediators such as IL-6 (FIG. 10A-10C) or GM-CSF (FIG. 11A-11C). There was no impact on other cytokines and chemokines involved in acute inflammation, myeloid-derived suppressor cells, and Th2 responses. The bispecific agonists were well tolerated. The activity of DGL620 in cynomolgus monkeys may not be representative of its activity in humans due to poor cross-reactivity against cyno IL-18Rβ. DGL620 is fully cross-reactive to IL-18Rα.

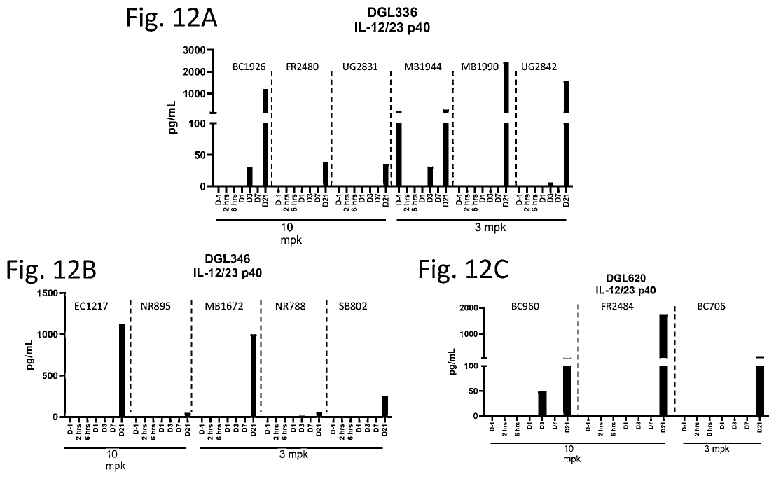

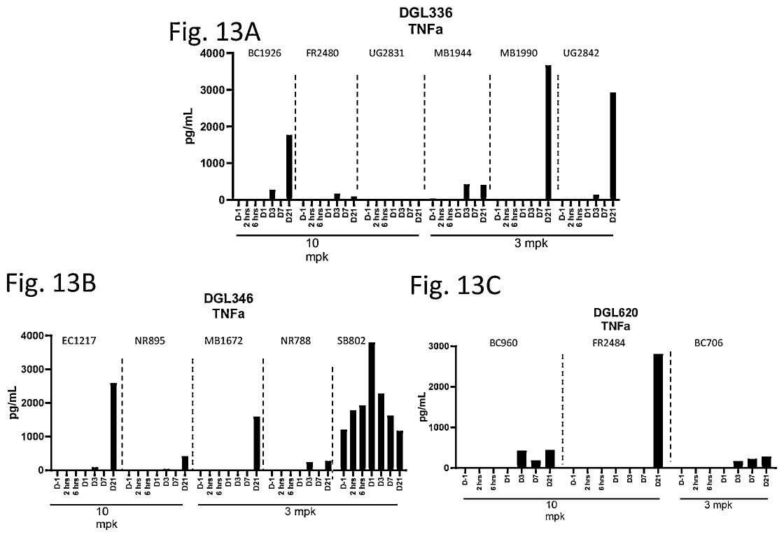

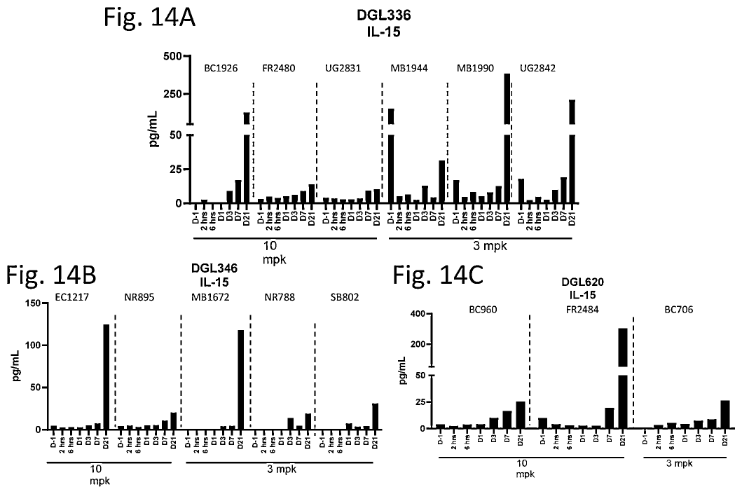

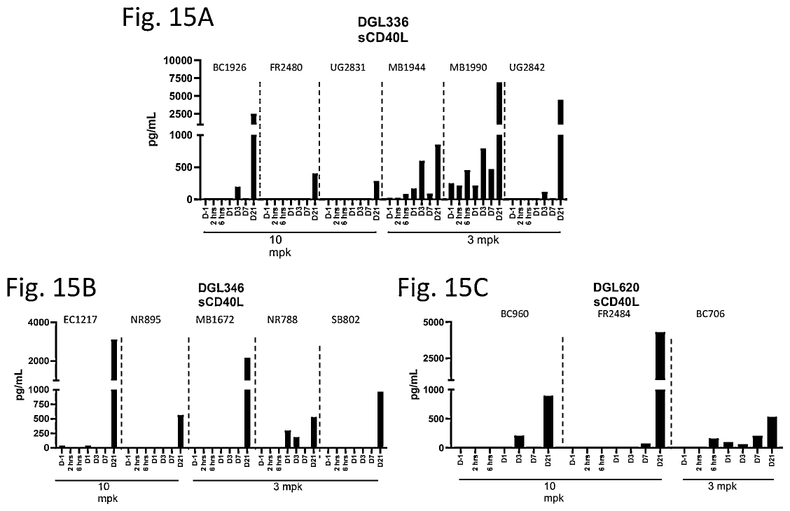

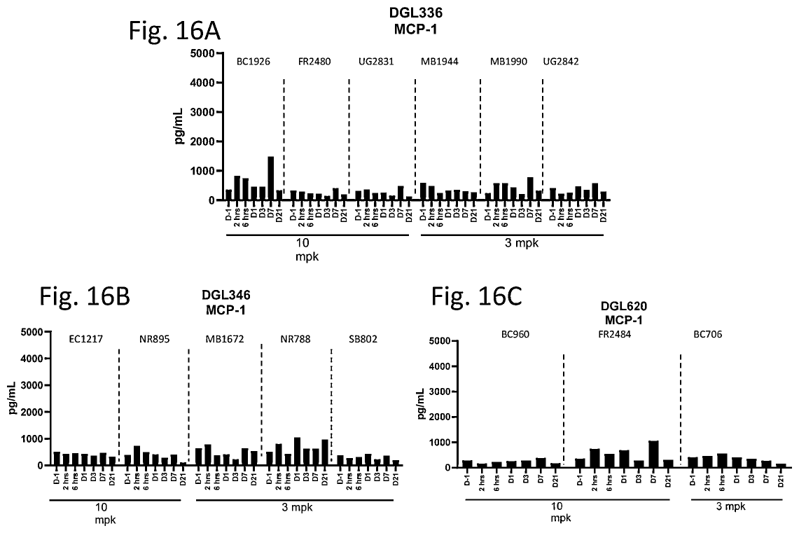

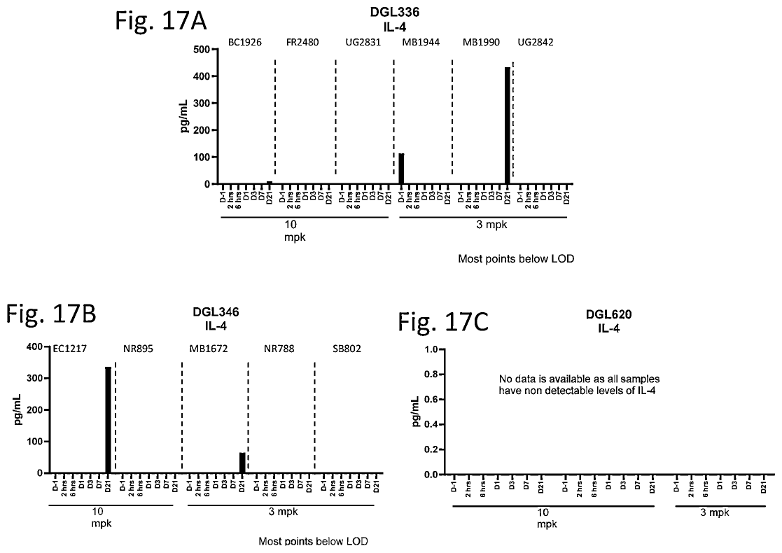

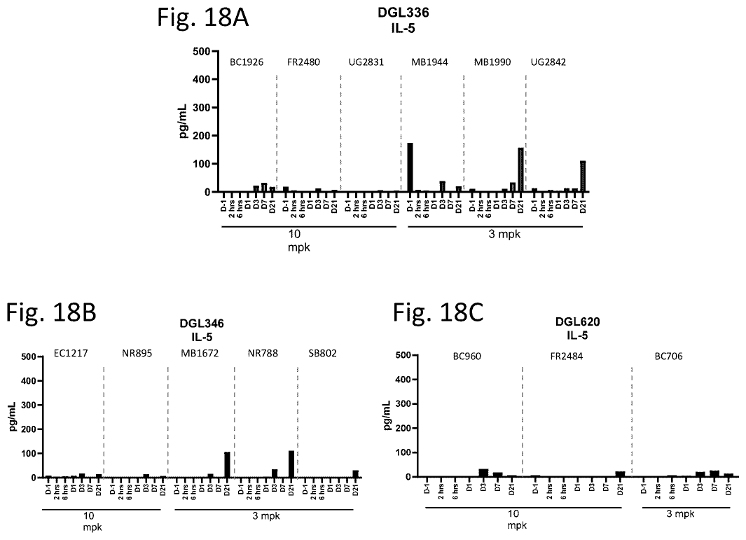

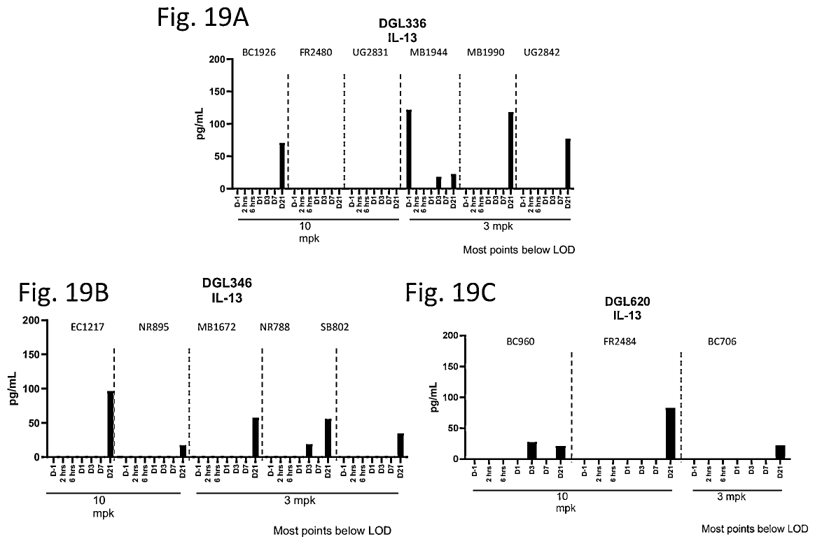

Additional factor measured were IL-12/23 p40 (FIG. 12A-12C), TNFα (FIG. 13A-13C), IL-15 (FIG. 14A-14C), sCD40L (FIG. 15A-15C), MCP-1 (FIG. 16A-16C), IL-4 (FIG. 17A-17C), IL-5 (FIG. 18A-18C), and IL-13 (FIG. 19A-19C).

### Example 8. Expression of Agonistic IL-18R Antibodies

DGL336, DGL346 and DGL620 were transiently transfected into 10 L of CHO-K1 cells using the WuXian Express Transfection platform (WuXi Biologics). Antibodies were purified using MabSelect SuRe (Cytiva) and polished using POROS XS (Thermo) and/or CaptoMMC ImpRes (Cytiva) depending on purity. Purity at each step was analyzed using SEC-HPLC and SDS-PAGE and confirmed using mass spectrometry. Final product yield for each are as follows: DGL336-1 g/L; DGL346-1.3 g/L; DGL620-0.8 g/L (Table 13).

TABLE 13
Quantification of expression of IL-18R antibodies from CHO K1 cells.

| | DGL336 | | DGL346 | | DGL620 | |
|---|---|---|---|---|---|---|
| Expression | 2.2 | g/L | 3.0 | g/L | 2.1 | g/L |
| (CHO-K1) | | | | | | |
| Purity (SEC-HPLC) | 99% | | 98.1% | | 94.7% | |
| Concentration | 21.1 | mg/ml | 22.1 | mg/ml | 25.7 | mg/ml |
| (mg/ml) | | | | | | |
| Formulation buffer | PBS | | PBS | | PBS | |
| Final Product Yield | 1 | g/L | 1.3 | g/L | 0.8 | g/L |

1. A multispecific binding protein comprising at least a first binding moiety which binds specifically to human interleukin-18 receptor α (IL-18Rα) and at least a second binding moiety which binds specifically to human interleukin-18 receptor β (IL-18Rβ), wherein:
a. the first binding moiety comprises an amino acid sequence of SEQ ID NO: 16 or SEQ ID NO: 17; and
b. the second binding moiety comprises an amino acid sequence of SEQ ID NO: 24, SEQ ID NO: 25 or SEQ ID NO: 32.

2. The multispecific binding protein of claim 1, wherein the first binding moiety and the second binding moiety are VHH domains.

3. The multispecific binding protein of claim 1, wherein the first and the second binding moieties are on the same polypeptide or are on different peptides.

4-5. (canceled)

6. The multispecific binding protein of claim 1, further comprising one or more modified hinge regions,
optionally wherein the upper hinge region of the first and the second modified hinge regions are the same sequence;
optionally wherein the upper hinge region of the first and the second modified hinge regions are different sequences;
optionally wherein the one or more hinges comprises:
i) an upper hinge region of up to 7 amino acids in length or is absent; and
ii) a middle hinge region and a lower hinge region, wherein the lower hinge region is linked to the N-terminus of a heavy chain constant region.

7-8. (canceled)

9. The multispecific binding protein of claim 6, wherein the upper hinge region comprises an amino acid sequence derived from an upper hinge region of a human IgG antibody, wherein the IgG antibody is selected from IgG1, IgG2, IgG3, and IgG4.

10-11. (canceled)

12. The multispecific binding protein of claim 6, wherein the upper hinge region is absent or comprises an amino acid sequence selected from SEQ ID NO: 2, SEQ ID NO: 4, and SEQ ID NO: 5.

13-15. (canceled)

16. The multispecific binding protein of claim 6, wherein the upper hinge is absent.

17. The multispecific binding protein of claim 1, further comprising all or part of an immunoglobulin Fc domain or variant thereof comprising a first Fc heavy chain and a second Fc heavy chain, optionally with reduced effector function.

18-25. (canceled)

26. The multispecific binding protein of claim 17, wherein at least one Fc heavy chain comprises one or more substitutions at amino acid positions 234, 235, or 237, according to EU numbering, optionally
wherein the substitution at amino acid position 234 is an alanine (A);
wherein the substitution at amino acid position 235 is an alanine (A); or
wherein the substitution at amino acid position 237 is an alanine (A).

27. (canceled)

28. The multispecific binding protein of claim 1, wherein the Fc domain comprises heterodimerization mutations to promote heterodimerization of the first binding moiety with the second binding moiety,
wherein the heterodimerization mutations are Knob-in-Hole (KIH) mutations;
optionally wherein the first Fc heavy chain comprises an amino acid substitution at position 366, 368, or 407 which produced a hole, and the second Fc heavy chain comprises an amino acid substitution at position 366 which produce a knob; or
optionally wherein the first Fc heavy chain comprises the amino acid substitution T366S, L368A, or Y407V, and the second Fc heavy chain comprises the amino acid substitution T366W.

29-31. (canceled)

32. The multispecific binding protein of claim 28, wherein the heterodimerization mutations are charge stabilization mutations,
optionally wherein the first Fc heavy chain comprises the amino acid substitution N297K, and the second Fc heavy chain comprises the amino acid substitution N297D, or
optionally wherein the first Fc heavy chain comprises the amino acid substitution T299K, and the second Fc heavy chain comprises the amino acid substitution T299D.

33-34. (canceled)

35. The multispecific binding protein of claim 28, wherein the heterodimerization mutations comprise an engineered disulfide bond,
optionally formed by a first Fc heavy chain comprising the amino acid substitution Y349C, and a second Fc heavy chain comprising the amino acid substitution S354C,
optionally wherein the engineered disulfide bond is formed by a C-terminal extension peptide fused to the C-terminus of each of the first Fc heavy chain and the second Fc heavy chain, optionally wherein the first Fc heavy chain C-terminal extension comprises the amino acid sequence GEC, and the second Fc heavy chain C-terminal extension comprises the amino acid sequence SCDKT (SEQ ID NO: 33).

36-39. (canceled)

40. The multispecific binding protein of claim 35, wherein at least one Fc domain comprises one or more mutations to promote increased half-life, or
wherein at least one Fc heavy chain comprises one or more substitutions at amino acid positions 252, 254, or 256, according to EU numbering, optionally
wherein the substitution at amino acid position 252 is a tyrosine (Y),
wherein the substitution at amino acid position 254 is a threonine (T), or
wherein the substitution at amino acid position 236 is a glutamic acid (E).

41. (canceled)

42. The multispecific binding protein of claim 1, wherein the first binding moiety which binds specifically to human IL-18Rα comprises an amino acid sequence set forth in SEQ ID NOs: 26, and the second binding moiety which binds specifically to human IL-18Rβ comprises an amino acid sequence set for the in any one of SEQ ID NOs: 27 or 28 or 31.

43. The multispecific binding protein of claim 1, wherein the binding protein comprises an amino acid sequence set forth in SEQ ID NO: 29.

44. The multispecific binding protein of claim 1, further comprising an Fc region comprising the amino acid sequence set forth in SEQ ID NO: 30.

45. The multispecific binding protein of claim 1, comprising
a) a first polypeptide chain comprising at least 80% identity to the amino acid sequence of SEQ ID NO: 26, and a second polypeptide chain comprising at least 80% identity to the amino acid sequence of SEQ ID NO: 28;
b) a first polypeptide chain comprising at least 80% identity to the amino acid sequence of SEQ ID NO: 29, and a second polypeptide chain comprising at least 80% identity to the amino acid sequence of SEQ ID NO: 30;
c) a first polypeptide chain comprising at least 80% identity to the amino acid sequence of SEQ ID NO: 26, and a second polypeptide chain comprising at least 80% identity to the amino acid sequence of SEQ ID NO: 27; or
d) a first polypeptide chain comprising at least 80% identity to the amino acid sequence of SEQ ID NO: 26, and a second polypeptide chain comprising at least 80% identity to the amino acid sequence of SEQ ID NO: 31.

46-48. (canceled)

49. A pharmaceutical composition comprising the multi-specific binding protein of claim 1 and a pharmaceutically acceptable carrier.

50. An isolated nucleic acid molecule encoding the multispecific binding protein of claim 1.

51. An expression vector comprising the nucleic acid molecule of claim 50.

52. A host cell comprising the expression vector of claim 51.

53-54. (canceled)

55. A method of stimulating IL-18R-mediated IFN-gamma expression in a subject, the method comprising administering to the multi-specific binding protein according to claim 1,
optionally wherein the multispecific binding protein stimulates anti-tumor cytokine production in the subject without substantially stimulating MCP-1 production, GM-CSF production, or production of markers of acute inflammatory or Th2 response in the subject relative to IL-18 stimulation of MCP-1 production, GM-CSF production, or production of markers of acute inflammatory or Th2 response;
optionally wherein the anti-tumor cytokines are selected from the group consisting of IFN-gamma, IL-2, IL-12, IL-15, CD40L, and TNFα;
optionally wherein the markers of acute inflammatory or Th2 response are selected from the group consisting of IL-6, IL-1β, IL-8, IL-4, IL-5, and IL-13;
optionally wherein the multispecific binding protein stimulates production of markers of acute inflammatory or Th2 response at least 5-fold less, at least 10-fold less, at least 50-fold less, or at least 100-fold less than IL-18;
optionally wherein the multispecific binding protein stimulates production of GM-CSF in the subject at least 5-fold less, at least 10-fold less, at least 50-fold less, or at least 100-fold less than IFN-gamma production in the subject.

56-60. (canceled)
